SEQUENCE LISTING (111 sequences)

SEQ ID NO: 1 - DNA, 24 bp, Artificial sequence
  Anti-sense primer HuCkappa
acactctcccctgttgaagctctt

SEQ ID NO: 2 - DNA, 23 bp, Artificial sequence
  Anti-sense primer HuClambda2
tgaacattctgtaggggccactg

SEQ ID NO: 3 - DNA, 23 bp, Artificial sequence
  Anti-sense primer HuClambda7
agagcattctgcaggggccactg

SEQ ID NO: 4 - DNA, 4941 bp, Artificial sequence
  Vector PDV-C06
aagcttgcatgcaaattctatttcaaggagacagtcataatgaaatacctattgcctacggcagccgctggattgttattactcgcggcccagccggccatggccgaggtgtttgactaatggggcgcgcctcagggaaccctggtcaccgtctcgagcggtacgggcggttcaggcggaaccggcagcggcactggcgggtcgacggaaattgtgctcacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggctttcggcggagggaccaaggtggagatcaaacgtgcggccgcacatcatcatcaccatcacggggccgcatataccgatattgaaatgaaccgcctgggcaaaggggccgcatagactgttgaaagttgtttagcaaaacctcatacagaaaattcatttactaacgtctggaaagacgacaaaactttagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 5 - DNA, 24 bp, Artificial sequence
  Anti-sense primer HuCIgG
gtccaccttggtgttgctgggctt

SEQ ID NO: 6 - protein, 669 aa, West Nile virus
MVTLSNFQGKVMMTVNATDVTDVITIPTAAGKNLCIVRAMDVGYMCDDTITYECPVLSAGNDPEDIDCWCTKSAVYVRYGRCTKTRHSRRSRRSLTVQTHGESTLANKKGAWMDSTKATRYLVKTESWILRNPGYALVAAVIGWMLGSNTMQRVVFVVLLLLVAPAYSFNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 7 - DNA, 23 bp, Artificial sequence
  Primer CMV-Spe
ccatgttgacattgattattgac

SEQ ID NO: 8 - DNA, 26 bp, Artificial sequence
  Primer WNV-E-95 REV
gctctagacttgccgatgctgctgcc

SEQ ID NO: 9 - DNA, 40 bp, Artificial sequence
  Primer clefsmaquwnv
ggaattcagcatggcccaggtgaccctgagcaacttccag

SEQ ID NO: 10 - DNA, 26 bp, Artificial sequence
  Primer reverse WNVmychis
gactagtcaactcaatggtgatggtg

SEQ ID NO: 11 - DNA, 6760 bp, Artificial sequence
  Vector pSyn-C18-HCgamma1
ctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaagtgataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagctggcctggatggcctgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatggcctgccccggcttcctgtgggccctggtgatcagcacctgcctgg

SEQ ID NO: 12 - DNA, 6283 bp, Artificial sequence
  Vector pSyn-C04-Clambda
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgttaattaacatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaatcgatgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatggcctgccccggcttcctgtgggccctggtgatcagcacctgcctcgagatccccggaccgcggccgcaagcttaccgtgctgggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagctaatagacttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 6267 bp, Artificial sequence
  Vector pSyn-C05-Ckappa
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgttaattaacatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaatcgatgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatggcctgccccggcttcctgtgggccctggtgatcagcacctgcctcgagttcagcggccctaagcggaccgtggccgctcccagcgtgttcatcttccccccctccgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggactccacctacagcctgagcagcaccctcaccctgagcaaggccgactacgagaagcacaaggtgtacgcctgcgaggtgacccaccagggcctgagcagccccgtgaccaagagcttcaaccggggcgagtgttaatagacttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccacaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 14 - DNA, 10515 bp, Artificial sequence
  Vector pIg-C911-HCgamma1
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaagctggcctggatatcctgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatgggatggagctgtatcatcctcttcttggtactgctgctggcccagccggccagtgaccttgaccggtgcaccacttttgatgatgttcaagctcctaattacactcaacatacttcatctatgaggggggtttactatcctgatgaaatttttagatcggacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaggcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgattttggcgacatttcaggcattaacgcttctgtcgtcaacattcaaaaagaaattgaccgcctcaatgaggtcgctaaaaatttaaatgaatcactcattgaccttcaagaactgggaaaatatgagcaatatattaaatggcctctcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgagtgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaagtgataatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 15 - DNA, 8792 bp, Artificial sequence
  Vector pIg-C910-Clambda
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgttaattaacatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaatcgatgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatgcggttctccgctcagctgctgggccttctggtgctgtggattcccggcgtctcgagatctatcgatgcatgccatggtaccaagcttgccaccatgagcagcagctcttggctgctgctgagcctggtggccgtgacagccgcccagagcaccatcgaggagcaggccaagaccttcctggacaagttcaaccacgaggccgaggacctgttctaccagagcagcctggccagctggaactacaacaccaacatcaccgaggagaacgtgcagaacatgaacaacgccggcgacaagtggagcgccttcctgaaggagcagagcacactggcccagatgtaccccctgcaggagatccagaacctgaccgtgaagctgcagctgcaggccctgcagcagaacggcagcagcgtgctgagcgaggacaagagcaagcggctgaacaccatcctgaacaccatgtccaccatctacagcaccggcaaagtgtgcaaccccgacaacccccaggagtgcctgctgctggagcccggcctgaacgagatcatggccaacagcctggactacaacgagcggctgtgggcctgggagagctggcggagcgaagtgggcaagcagctgcggcccctgtacgaggagtacgtggtgctgaagaacgagatggccagggccaaccactacgaggactacggcgactactggagaggcgactacgaagtgaacggcgtggacggctacgactacagcagaggccagctgatcgaggacgtggagcacaccttcgaggagatcaagcctctgtacgagcacctgcacgcctacgtgcgggccaagctgatgaacgcctaccccagctacatcagccccatcggctgcctgcccgcccacctgctgggcgacatgtggggccggttctggaccaacctgtacagcctgaccgtgcccttcggccagaagcccaacatcgacgtgaccgacgccatggtggaccaggcctgggacgcccagcggatcttcaaggaggccgagaagttcttcgtgagcgtgggcctgcccaacatgacccagggcttttgggagaacagcatgctgaccgaccccggcaatgtgcagaaggccgtgtgccaccccaccgcctgggacctgggcaagggcgacttccggatcctgatgtgcaccaaagtgaccatggacgacttcctgaccgcccaccacgagatgggccacatccagtacgacatggcctacgccgcccagcccttcctgctgcggaacggcgccaacgagggctttcacgaggccgtgggcgagatcatgagcctgagcgccgccacccccaagcacctgaagagcatcggcctgctgagccccgacttccaggaggacaacgagaccgagatcaacttcctgctgaagcaggccctgaccatcgtgggcaccctgcccttcacctacatgctggagaagtggcggtggatggtgtttaagggcgagatccccaaggaccagtggatgaagaagtggtgggagatgaagcgggagatcgtgggcgtggtggagcccgtgccccacgacgagacctactgcgaccccgccagcctgttccacgtgagcaacgactactccttcatccggtactacacccggaccctgtaccagttccagttccaggaggccctgtgccaggccgccaagcacgagggccccctgcacaagtgcgacatcagcaacagcaccgaggccggacagaaactgttcaacatgctgcggctgggcaagagcgagccctggaccctggccctggagaatgtggtgggcgccaagaacatgaatgtgcgccccctgctgaactacttcgagcccctgttcacctggctgaaggaccagaacaagaacagcttcgtgggctggagcaccgactggagcccctacgccgaccagagcatcaaagtgcggatcagcctgaagagcgccctgggcgacaaggcctacgagtggaacgacaacgagatgtacctgttccggagcagcgtggcctatgccatgcggcagtacttcctgaaagtgaagaaccagatgatcctgttcggcgaggaggacgtgagagtggccaacctgaagccccggatcagcttcaacttcttcgtgaccgcccccaagaacgtgagcgacatcatcccccggaccgaagtggagaaggccatccggatgagccggagccggatcaacgacgccttccggctgaacgacaactccctggagttcctgggcatccagcccaccctgggccctcccaaccagccccccgtgagcatctggctgatcgtgtttggcgtggtgatgggcgtgatcgtggtgggaatcgtgatcctgatcttcaccggcatccgggaccggaagaagaagaacaaggcccggagcggcgagaacccctacgccagcatcgatatcagcaagggcgagaacaaccccggcttccagaacaccgacgacgtgcagaccagcttctgataatctagaacgagctcgaattcgaagcttctgcagacgcgtcgacgtcatatggatccgatatcgccgtggcggccgcaggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagctaatagacttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 16 - DNA, 8777 bp, Artificial sequence
  Vector pIg-C909-Ckappa
tcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgttaattaacatgaagaatctgcttagggttaggcgttttgcgctgcttcgctaggtggtcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaatcgatgactctcttaggtagccttgcagaagttggtcgtgaggcactgggcaggtaagtatcaaggttacaagacaggtttaaggagatcaatagaaactgggcttgtcgagacagagaagactcttgcgtttctgataggcacctattggtcttactgacatccactttgcctttctctccacaggtgtccactcccagttcaattacagctcgccaccatgcggctgcccgcccagctgctgggccttctcatgctgtgggtgcccgcctcgagatctatcgatgcatgccatggtaccaagcttgccaccatgagcagcagctcttggctgctgctgagcctggtggccgtgacagccgcccagagcaccatcgaggagcaggccaagaccttcctggacaagttcaaccacgaggccgaggacctgttctaccagagcagcctggccagctggaactacaacaccaacatcaccgaggagaacgtgcagaacatgaacaacgccggcgacaagtggagcgccttcctgaaggagcagagcacactggcccagatgtaccccctgcaggagatccagaacctgaccgtgaagctgcagctgcaggccctgcagcagaacggcagcagcgtgctgagcgaggacaagagcaagcggctgaacaccatcctgaacaccatgtccaccatctacagcaccggcaaagtgtgcaaccccgacaacccccaggagtgcctgctgctggagcccggcctgaacgagatcatggccaacagcctggactacaacgagcggctgtgggcctgggagagctggcggagcgaagtgggcaagcagctgcggcccctgtacgaggagtacgtggtgctgaagaacgagatggccagggccaaccactacgaggactacggcgactactggagaggcgactacgaagtgaacggcgtggacggctacgactacagcagaggccagctgatcgaggacgtggagcacaccttcgaggagatcaagcctctgtacgagcacctgcacgcctacgtgcgggccaagctgatgaacgcctaccccagctacatcagccccatcggctgcctgcccgcccacctgctgggcgacatgtggggccggttctggaccaacctgtacagcctgaccgtgcccttcggccagaagcccaacatcgacgtgaccgacgccatggtggaccaggcctgggacgcccagcggatcttcaaggaggccgagaagttcttcgtgagcgtgggcctgcccaacatgacccagggcttttgggagaacagcatgctgaccgaccccggcaatgtgcagaaggccgtgtgccaccccaccgcctgggacctgggcaagggcgacttccggatcctgatgtgcaccaaagtgaccatggacgacttcctgaccgcccaccacgagatgggccacatccagtacgacatggcctacgccgcccagcccttcctgctgcggaacggcgccaacgagggctttcacgaggccgtgggcgagatcatgagcctgagcgccgccacccccaagcacctgaagagcatcggcctgctgagccccgacttccaggaggacaacgagaccgagatcaacttcctgctgaagcaggccctgaccatcgtgggcaccctgcccttcacctacatgctggagaagtggcggtggatggtgtttaagggcgagatccccaaggaccagtggatgaagaagtggtgggagatgaagcgggagatcgtgggcgtggtggagcccgtgccccacgacgagacctactgcgaccccgccagcctgttccacgtgagcaacgactactccttcatccggtactacacccggaccctgtaccagttccagttccaggaggccctgtgccaggccgccaagcacgagggccccctgcacaagtgcgacatcagcaacagcaccgaggccggacagaaactgttcaacatgctgcggctgggcaagagcgagccctggaccctggccctggagaatgtggtgggcgccaagaacatgaatgtgcgccccctgctgaactacttcgagcccctgttcacctggctgaaggaccagaacaagaacagcttcgtgggctggagcaccgactggagcccctacgccgaccagagcatcaaagtgcggatcagcctgaagagcgccctgggcgacaaggcctacgagtggaacgacaacgagatgtacctgttccggagcagcgtggcctatgccatgcggcagtacttcctgaaagtgaagaaccagatgatcctgttcggcgaggaggacgtgagagtggccaacctgaagccccggatcagcttcaacttcttcgtgaccgcccccaagaacgtgagcgacatcatcccccggaccgaagtggagaaggccatccggatgagccggagccggatcaacgacgccttccggctgaacgacaactccctggagttcctgggcatccagcccaccctgggccctcccaaccagccccccgtgagcatctggctgatcgtgtttggcgtggtgatgggcgtgatcgtggtgggaatcgtgatcctgatcttcaccggcatccgggaccggaagaagaagaacaaggcccggagcggcgagaacccctacgccagcatcgatatcagcaagggcgagaacaaccccggcttccagaacaccgacgacgtgcagaccagcttctgataatctagaacgagctcgaattcgaagcttctgcagacgcgtcgacgtcatatggatccgatatcgccgtggcggccgcacccagcgtgttcatcttccccccctccgacgagcagctgaagagcggcaccgccagcgtggtgtgcctgctgaacaacttctacccccgggaggccaaggtgcagtggaaggtggacaacgccctgcagagcggcaacagccaggagagcgtgaccgagcaggacagcaaggactccacctacagcctgagcagcaccctcaccctgagcaaggccgactacgagaagcacaaggtgtacgcctgcgaggtgacccaccagggcctgagcagccccgtgaccaagagcttcaaccggggcgagtgttaatagacttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggccatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccacaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacg

SEQ ID NO: 17 - DNA, 47 bp, Artificial sequence
  Oligonucleotide 5H-A
acctgtcttgaattctccatggcccaggtgcagctggtgcagtctgg

SEQ ID NO: 18 - DNA, 47 bp, Artificial sequence
  Oligonucleotide SY3H-A
gcccttggtgctagcgctggagacggtcaccagggtgccctggcccc

SEQ ID NO: 19 - DNA, 47 bp, Artificial sequence
  Oligonucleotide SY3H-C
gcccttggtgctagcgctggagacggtcacggtggtgccctggcccc

SEQ ID NO: 20 - DNA, 47 bp, Artificial sequence
  Oligonucleotide 5H-C
acctgtcttgaattctccatggcccaggtgcagctggtggagtctgg

SEQ ID NO: 21 - DNA, 52 bp, Artificial sequence
  Oligonucleotide 5L-C
acctgtctcgagttttccatggctcagtccgtgctgacccagcctccctcag

SEQ ID NO: 22 - DNA, 43 bp, Artificial sequence
  Oligonucleotide sy3L-Amod
ccagcacggtaagcttcagcacggtcaccttggtgccagttcc

SEQ ID NO: 23 - DNA, 28 bp, Artificial sequence
  Oligonucleotide SY3L-C
ccagcacggtaagcttggtgcctccgcc

SEQ ID NO: 24 - DNA, 1344 bp, Artificial sequence
  Heavy chain CR4261
caggtgcagctggtgcagtctggggttgaggtgaagaggcctggggcctcagtgaaggtctcctgcaaggcttctggatacaccttcaccagttataccttacattgggtgcgccaggcccccggacaaaggcctgagtggatgggatggatccaccctgtcaatggtgacacaaaatattcacagaagttccagggcagagtcaccattaccagggacacatccgcgagcacagcctacatggagctgagcagcctgagatctgaagacacggctgtgtattactgtgcgagggggtacgacagctggtcttttgactactggggccagggcaccctggtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 25 - protein, 448 aa, Artificial sequence
  Heavy chain CR4261
QVQLVQSGVEVKRPGASVKVSCKASGYTFTSYTLHWVRQAPGQRPEWMGWIHPVNGDTKYSQKFQGRVTITRDTSASTAYMELSSLRSEDTAVYYCARGYDSWSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 1377 bp, Artificial sequence
  Heavy chain CR4267
caggtgcagctggtgcagtctgggactgaggtgaagaagcctgggtcctcggtgaaggtctcctgcaaggcttctggaggcaccttcagcaactatgctatcagctgggtgcgacaggcccctggacaagggcttgagtggatgggaggtatcgtccctaagtttgctacaacaagctacgcacagaggttccagggcagagtcacgattaccgcggacgaatccacgagcacagtctacatggagctgagcagcctgagatctgaggacacggccgtgtatttttgtgcgagatttggagtggtggtagctgccgaccgtcaaggagcctactactactacggtatggacgtctggggccagggcaccaccgtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 27 - protein, 459 aa, Artificial sequence
  Heavy chain CR4267
QVQLVQSGTEVKKPGSSVKVSCKASGGTFSNYAISWVRQAPGQGLEWMGGIVPKFATTSYAQRFQGRVTITADESTSTVYMELSSLRSEDTAVYFCARFGVVVAADRQGAYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1344 bp, Artificial sequence
  Heavy chain CR4328
caggtgcagctggtgcagtctggggctgaaatgaagaagcctggggcctcagtgaaggtctcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagggggtacgacagctggtcttttgactactggggccagggcaccctggtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 29 - protein, 448 aa, Artificial sequence
  Heavy chain CR4328
QVQLVQSGAEMKKPGASVKVSCKASGYTFTSYAMHWVRQAPGQRLEWMGWINAGNGNTKYSQKFQGRVTITRDTSASTAYMELSSLRSEDTAVYYCARGYDSWSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 1347 bp, Artificial sequence
  Heavy chain CR4335
caggtgcagctggtggagtctgggggaggcgtggtccagcctggcaggtccctgagactctcctgtgtagcctctggattcaccttcagtaaggacgctatgcactgggtccgccaggctccaggcaaggggctagagtgggtggcagttatatcatatgatggaagtgataaacactacgcagactccgtgaagggccgagtcaccatctccagagacaattccaggaaaacgctgcatctgcgaatggacagcctgagagctgaggacacggctctatattactgtgcgagaggatacaactctggtcattactttgactactggggccagggcaccctggtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 31 - protein, 449 aa, Artificial sequence
  Heavy chain CR4335
QVQLVESGGGVVQPGRSLRLSCVASGFTFSKDAMHWVRQAPGKGLEWVAVISYDGSDKHYADSVKGRVTISRDNSRKTLHLRMDSLRAEDTALYYCARGYNSGHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1359 bp, Artificial sequence
  Heavy chain CR4354
caggtgcagctggtgcagtctggggctgaggtgaggaagcctggggcctcagtgaaggtttcctgcaaggcatctggatacaccttcacccactattatatgcactgggtgcgacaggcccctggacaagggcttgagtggatgggaataatcaaccctagtggtggtagcacaacctacgcacagaagctccagggcagagtcaccatgaccagggacacgtccacgagcacagtctacatggagctgagcagcctgagatctgaggacacggccgtgtattactgtgcgagagattggggctccaattacgtttgggggagttatcccaagtactggggccagggcaccctggtgaccgtctccagcgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 33 - protein, 453 aa, Artificial sequence
  Heavy chain CR4354
QVQLVQSGAEVRKPGASVKVSCKASGYTFTHYYMHWVRQAPGQGLEWMGIINPSGGSTTYAQKLQGRVTMTRDTSTSTVYMELSSLRSEDTAVYYCARDWGSNYVWGSYPKYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - DNA, 1368 bp, Artificial sequence
  Heavy chain CR4383
caggtgcagctggtgcagtctggggctgaggtgaagaagcctggggcctcagtgaaggtctcctgcaagacttctggatacaccttcaccgactactatctgcactgggtgcgacacgcccctggacaagggcttgagtggatgggatggatcaaccctaacagtggtgacacaaactatgcacagaagtttcagggcagggtcaccatgaccagggacacgtccatcagcacagcctacatggacctgagcaggctgagatctgacgacgcggccgtgtattactgtgcgagagattggaatacggtgactacgtactactattactactacggtatggacgtctggggccaagggaccacggtcaccgtctcgagtgctagcaccaagggccccagcgtgttccccctggcccccagcagcaagagcaccagcggcggcacagccgccctgggctgcctggtgaaggactacttccccgagcccgtgaccgtgagctggaacagcggcgccttgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaaacgcgtggagcccaagagctgcgacaagacccacacctgccccccctgccctgcccccgagctgctgggcggaccctccgtgttcctgttcccccccaagcccaaggacaccctcatgatcagccggacccccgaggtgacctgcgtggtggtggacgtgagccacgaggaccccgaggtgaagttcaactggtacgtggacggcgtggaggtgcacaacgccaagaccaagccccgggaggagcagtacaacagcacctaccgggtggtgagcgtgctcaccgtgctgcaccaggactggctgaacggcaaggagtacaagtgcaaggtgagcaacaaggccctgcctgcccccatcgagaagaccatcagcaaggccaagggccagccccgggagccccaggtgtacaccctgccccccagccgggaggagatgaccaagaaccaggtgtccctcacctgtctggtgaagggcttctaccccagcgacatcgccgtggagtgggagagcaacggccagcccgagaacaactacaagaccaccccccctgtgctggacagcgacggcagcttcttcctgtacagcaagctcaccgtggacaagagccggtggcagcagggcaacgtgttcagctgcagcgtgatgcacgaggccctgcacaaccactacacccagaagagcctgagcctgagccccggcaag

SEQ ID NO: 35 - protein, 456 aa, Artificial sequence
  Heavy chain CR4383
QVQLVQSGAEVKKPGASVKVSCKTSGYTFTDYYLHWVRHAPGQGLEWMGWINPNSGDTNYAQKFQGRVTMTRDTSISTAYMDLSRLRSDDAAVYYCARDWNTVTTYYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - DNA, 669 bp, Artificial sequence
  Light chain CR4261
tcgacgcaggctgtgctgactcagccgtcctcagcgtctgggacccccgggcagagggtcaccatctcttgttctggaagcagccccaacatcggaagtaataatgtaaactggtaccagcagctcccaggaacggcccccaaactcctcatttatagtaataatcagcggccctcaggggtccctggccgattctctggctccaagtctggcacctcagcctccctggccatcagtgggctccagtctgaggatgaggctgattattactgtgcagcatgggatgacggcctaagtggtaattatgtcttcggagctgggacccagctcaccgttttaagtgcggccgcaggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagc

SEQ ID NO: 37 - protein, 223 aa, Artificial sequence
  Light chain CR4261
STQAVLTQPSSASGTPGQRVTISCSGSSPNIGSNNVNWYQQLPGTAPKLLIYSNNQRPSGVPGRFSGSKSGTSASLAISGLQSEDEADYYCAAWDDGLSGNYVFGAGTQLTVLSAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - DNA, 648 bp, Artificial sequence
  Light chain CR4267
cagtccgtgctgacccagcctccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattattgtgcagcttgggatgacaccctgaatggttatgtcttcggaactggcaccaagcttaccgtgctgggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagc

SEQ ID NO: 39 - protein, 216 aa, Artificial sequence
  Light chain CR4267
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDTLNGYVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - DNA, 666 bp, Artificial sequence
  Light chain CR4328
tcgacgcaggctgtgctgactcagccgtcctcagtgtctgggacccccgggcagagggtcaccatctcttgttctggaagcagctccaacatcggaagtaatactgtaaactggtaccagcagcttccaggaacagcccccaaactcctcatctatggtaacaacaatcggccctcaggggtccctgcccgattctctggctccaggtctggcacctcagcctccctggccatcagtgggctccggtccgaggatgaggctgattattactgtgcagcatgggatgacagcctgaatggcccggtgttcggcggagggaccaagctgaccgtcctaggtgcggccgcaggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagc

SEQ ID NO: 41 - protein, 222 aa, Artificial sequence
  Light chain CR4328
STQAVLTQPSSVSGTPGQRVTISCSGSSSNIGSNTVNWYQQLPGTAPKLLIYGNNNRPSGVPARFSGSRSGTSASLAISGLRSEDEADYYCAAWDDSLNGPVFGGGTKLTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - DNA, 666 bp, Artificial sequence
  Light chain CR4335
tcgacgcagtctgtgctgactcagccaccctcaacgtctgggacccccgggcagagggtcaccatctcttgttctggaagcgactccaacatcggcagtaatactgtaaactggtaccagcagctcccaggaatggcccccaaactcctcatctataggaataatcagcggccctcaggggtccctgaccgattctctggctccaagtctggcacctcagcctccctggccatcagtggtctccagtctgaagatgaggctgactatttctgtgcatcatgggatgccaatctgggtggtccgctgttcggtggggggaccaaggtcaccgtcctaggtgcggccgcaggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagc

SEQ ID NO: 43 - protein, 222 aa, Artificial sequence
  Light chain CR4335
STQSVLTQPPSTSGTPGQRVTISCSGSDSNIGSNTVNWYQQLPGMAPKLLIYRNNQRPSGVPDRFSGSKSGTSASLAISGLQSEDEADYFCASWDANLGGPLFGGGTKVTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - DNA, 648 bp, Artificial sequence
  Light chain CR4354
cagtccgtgctgacccagcctccctcagtgtctgcggcccccggacagaaggtcaccatctcctgttctggaagcagctccaacatcggaagtaatactgtaaactggtaccagcagctcccaggaacggcccccaaactcctcatctatagtaataatcagcggccctcagggatccctgaccgattctctggctccaagtctggcacctcagcctccctggccatcagtgggctccaatctgaagatgaggctgattattactgtgcagcatgggatgacagcctgaatggtccggtgttcggcggaggcaccaagcttaccgtgctgggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagc

SEQ ID NO: 45 - protein, 216 aa, Artificial sequence
  Light chain CR4354
QSVLTQPPSVSAAPGQKVTISCSGSSSNIGSNTVNWYQQLPGTAPKLLIYSNNQRPSGIPDRFSGSKSGTSASLAISGLQSEDEADYYCAAWDDSLNGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - DNA, 666 bp, Artificial sequence
  Light chain CR4383
tcgacgcagtctgtgctgactcagccaccctcagcgtctgggacccccgggcagagggtcaccatctcttgttctggaggcatctccaacatcggaagtaatagtgtaaattggttccagcaactcccaggaacggcccccaaactcctcctctacagtaataatcagcgggcctcaggggtccctgaccgattctctggctccaagtctggcacctcagcctccctggccatcagtgggctccagtctgaggatgaggttgattattactgtgcagcatgggatgaccgcctgattggttatgtcttcggaacggggaccaaggtcaccgtcctaggtgcggccgcaggccagcccaaggccgctcccagcgtgaccctgttccccccctcctccgaggagctgcaggccaacaaggccaccctggtgtgcctcatcagcgacttctaccctggcgccgtgaccgtggcctggaaggccgacagcagccccgtgaaggccggcgtggagaccaccacccccagcaagcagagcaacaacaagtacgccgccagcagctacctgagcctcacccccgagcagtggaagagccaccggagctacagctgccaggtgacccacgagggcagcaccgtggagaagaccgtggcccccaccgagtgcagc

SEQ ID NO: 47 - protein, 222 aa, Artificial sequence
  Light chain CR4383
STQSVLTQPPSASGTPGQRVTISCSGGISNIGSNSVNWFQQLPGTAPKLLLYSNNQRASGVPDRFSGSKSGTSASLAISGLQSEDEVDYYCAAWDDRLIGYVFGTGTKVTVLGAAAGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - DNA, 23 bp, Artificial sequence
  Primer HuVlambda1A
cagtctgtgctgactcagccacc

SEQ ID NO: 49 - DNA, 23 bp, Artificial sequence
  Primer HuVlambda1B
cagtctgtgytgacgcagccgcc

SEQ ID NO: 50 - DNA, 23 bp, Artificial sequence
  Primer HuVlambda1C
cagtctgtcgtgacgcagccgcc

SEQ ID NO: 51 - DNA, 21 bp, Artificial sequence
  Primer HuVlambda2
cartctgccctgactcagcct

SEQ ID NO: 52 - DNA, 23 bp, Artificial sequence
  Primer HuVlambda3A
tcctatgwgctgactcagccacc

SEQ ID NO: 53 - DNA, 23 bp, Artificial sequence
  Primer HuVlambda3B
tcttctgagctgactcaggaccc

SEQ ID NO: 54 - DNA, 23 bp, Artificial sequence
  Primer HuVlambda4
cacgttatactgactcaaccgcc

SEQ ID NO: 55 - DNA, 23 bp, Artificial sequence
  Primer HuVlambda5
caggctgtgctgactcagccgtc

SEQ ID NO: 56 - DNA, 23 bp, Artificial sequence
  Primer HuVlambda6
aattttatgctgactcagcccca

SEQ ID NO: 57 - DNA, 23 bp, Artificial sequence
  Primer HuVlambda7/8
cagrctgtggtgacycaggagcc

SEQ ID NO: 58 - DNA, 23 bp, Artificial sequence
  Primer HuVlambda9
cwgcctgtgctgactcagccmcc

SEQ ID NO: 59 - DNA, 23 bp, Artificial sequence
  Primer HuVkappa1B
gacatccagwtgacccagtctcc

SEQ ID NO: 60 - DNA, 23 bp, Artificial sequence
  Primer HuVkappa2
gatgttgtgatgactcagtctcc

SEQ ID NO: 61 - DNA, 23 bp, Artificial sequence
  Primer HuVkappa3
gaaattgtgwtgacrcagtctcc

SEQ ID NO: 62 - DNA, 23 bp, Artificial sequence
  Primer HuVkappa4
gatattgtgatgacccacactcc

SEQ ID NO: 63 - DNA, 23 bp, Artificial sequence
  Primer HuVkappa5
gaaacgacactcacgcagtctcc

SEQ ID NO: 64 - DNA, 23 bp, Artificial sequence
  Primer HuVkappa6
gaaattgtgctgactcagtctcc

SEQ ID NO: 65 - DNA, 41 bp, Artificial sequence
  Primer HuVkappa1B-SalI
tgagcacacaggtcgacggacatccagwtgacccagtctcc

SEQ ID NO: 66 - DNA, 41 bp, Artificial sequence
  Primer HuVkappa2-SalI
tgagcacacaggtcgacggatgttgtgatgactcagtctcc

SEQ ID NO: 67 - DNA, 41 bp, Artificial sequence
  Primer HuVkappa3B-SalI
tgagcacacaggtcgacggaaattgtgwtgacrcagtctcc

SEQ ID NO: 68 - DNA, 41 bp, Artificial sequence
  Primer HuVkappa4B-SalI
tgagcacacaggtcgacggatattgtgatgacccacactcc

SEQ ID NO: 69 - DNA, 41 bp, Artificial sequence
  Primer HuVkappa5-SalI
tgagcacacaggtcgacggaaacgacactcacgcagtctcc

SEQ ID NO: 70 - DNA, 41 bp, Artificial sequence
  Primer HuVkappa6-SalI
tgagcacacaggtcgacggaaattgtgctgactcagtctcc

SEQ ID NO: 71 - DNA, 48 bp, Artificial sequence
  Primer HuJkappa1-NotI
gagtcattctcgacttgcggccgcacgtttgatttccaccttggtccc

SEQ ID NO: 72 - DNA, 48 bp, Artificial sequence
  Primer HuJkappa2-NotI
gagtcattctcgacttgcggccgcacgtttgatctccagcttggtccc

SEQ ID NO: 73 - DNA, 48 bp, Artificial sequence
  Primer HuJkappa3-NotI
gagtcattctcgacttgcggccgcacgtttgatatccactttggtccc

SEQ ID NO: 74 - DNA, 48 bp, Artificial sequence
  Primer HuJkappa4-NotI
gagtcattctcgacttgcggccgcacgtttgatctccaccttggtccc

SEQ ID NO: 75 - DNA, 48 bp, Artificial sequence
  Primer HuJkappa5-NotI
gagtcattctcgacttgcggccgcacgtttaatctccagtcgtgtccc

SEQ ID NO: 76 - DNA, 41 bp, Artificial sequence
  Primer HuVlambda1A-SalI
tgagcacacaggtcgacgcagtctgtgctgactcagccacc

SEQ ID NO: 77 - DNA, 41 bp, Artificial sequence
  Primer HuVlambda1B-SalI
tgagcacacaggtcgacgcagtctgtgytgacgcagccgcc

SEQ ID NO: 78 - DNA, 41 bp, Artificial sequence
  Primer HuVlambda1C-SalI
tgagcacacaggtcgacgcagtctgtcgtgacgcagccgcc

SEQ ID NO: 79 - DNA, 39 bp, Artificial sequence
  Primer HuVlambda2-SalI
tgagcacacaggtcgacgcartctgccctgactcagcct

SEQ ID NO: 80 - DNA, 41 bp, Artificial sequence
  Primer HuVlambda3A-SalI
tgagcacacaggtcgacgtcctatgwgctgactcagccacc

SEQ ID NO: 81 - DNA, 41 bp, Artificial sequence
  Primer HuVlambda3B-SalI
tgagcacacaggtcgacgtcttctgagctgactcaggaccc

SEQ ID NO: 82 - DNA, 41 bp, Artificial sequence
  Primer HuVlambda4-SalI
tgagcacacaggtcgacgcacgttatactgactcaaccgcc

SEQ ID NO: 83 - DNA, 41 bp, Artificial sequence
  Primer HuVlambda5-SalI
tgagcacacaggtcgacgcaggctgtgctgactcagccgtc

SEQ ID NO: 84 - DNA, 41 bp, Artificial sequence
  Primer HuVlambda6-SalI
tgagcacacaggtcgacgaattttatgctgactcagcccca

SEQ ID NO: 85 - DNA, 41 bp, Artificial sequence
  Primer HuVlambda7/8-SalI
tgagcacacaggtcgacgcagrctgtggtgacycaggagcc

SEQ ID NO: 86 - DNA, 41 bp, Artificial sequence
  Primer HuVlambda9-SalI
tgagcacacaggtcgacgcwgcctgtgctgactcagccmcc

SEQ ID NO: 87 - DNA, 48 bp, Artificial sequence
  Primer HuJlambda1-NotI
gagtcattctcgacttgcggccgcacctaggacggtgaccttggtccc

SEQ ID NO: 88 - DNA, 48 bp, Artificial sequence
  Primer HuJlambda2/3-NotI
gagtcattctcgacttgcggccgcacctaggacggtcagcttggtccc

SEQ ID NO: 89 - DNA, 48 bp, Artificial sequence
  Primer HuJlambda4/5-NotIv
gagtcattctcgacttgcggccgcacytaaaacggtgagctgggtccc

SEQ ID NO: 90 - DNA, 23 bp, Artificial sequence
  Primer HuVH1B/7A
cagrtgcagctggtgcartctgg

SEQ ID NO: 91 - DNA, 23 bp, Artificial sequence
  Primer HuVH1C
saggtccagctggtrcagtctgg

SEQ ID NO: 92 - DNA, 23 bp, Artificial sequence
  Primer HuVH2B
saggtgcagctggtggagtctgg

SEQ ID NO: 93 - DNA, 23 bp, Artificial sequence
  Primer HuVH3B
saggtgcagctggtggagtctgg

SEQ ID NO: 94 - DNA, 23 bp, Artificial sequence
  Primer HuVH3C
gaggtgcagctggtggagwcygg

SEQ ID NO: 95 - DNA, 23 bp, Artificial sequence
  Primer HuVH4B
caggtgcagctacagcagtgggg

SEQ ID NO: 96 - DNA, 23 bp, Artificial sequence
  Primer HuVH4C
cagstgcagctgcaggagtcsgg

SEQ ID NO: 97 - DNA, 23 bp, Artificial sequence
  Primer HuVH5B
gargtgcagctggtgcagtctgg

SEQ ID NO: 98 - DNA, 23 bp, Artificial sequence
  Primer HuVH6A
caggtacagctgcagcagtcagg

SEQ ID NO: 99 - DNA, 56 bp, Artificial sequence
  Primer HuVH1B/7A-SfiI
gtcctcgcaactgcggcccagccggccatggcccagrtgcagctggtgcartctgg

SEQ ID NO: 100 - DNA, 56 bp, Artificial sequence
  Primer HuVH1C-SfiI
gtcctcgcaactgcggcccagccggccatggccsaggtccagctggtrcagtctgg

SEQ ID NO: 101 - DNA, 56 bp, Artificial sequence
  Primer HuVH2B-SfiI
gtcctcgcaactgcggcccagccggccatggcccagrtcaccttgaaggagtctgg

SEQ ID NO: 102 - DNA, 56 bp, Artificial sequence
  Primer HuVH3B-SfiI
gtcctcgcaactgcggcccagccggccatggccsaggtgcagctggtggagtctgg

SEQ ID NO: 103 - DNA, 56 bp, Artificial sequence
  Primer HuVH3C-SfiI
gtcctcgcaactgcggcccagccggccatggccgaggtgcagctggtggagwcygg

SEQ ID NO: 104 - DNA, 56 bp, Artificial sequence
  Primer HuVH4B-SfiI
gtcctcgcaactgcggcccagccggccatggcccaggtgcagctacagcagtgggg

SEQ ID NO: 105 - DNA, 56 bp, Artificial sequence
  Primer HuVH4C-SfiI
gtcctcgcaactgcggcccagccggccatggcccagstgcagctgcaggagtcsgg

SEQ ID NO: 106 - DNA, 56 bp, Artificial sequence
  Primer HuVH5B-SfiI
gtcctcgcaactgcggcccagccggccatggccgargtgcagctggtgcagtctgg

SEQ ID NO: 107 - DNA, 56 bp, Artificial sequence
  Primer HuVH6A-SfiI
gtcctcgcaactgcggcccagccggccatggcccaggtacagctgcagcagtcagg

SEQ ID NO: 108 - DNA, 36 bp, Artificial sequence
  Primer HuJH1/2-XhoI
gagtcattctcgactcgagacggtgaccagggtgcc

SEQ ID NO: 109 - DNA, 36 bp, Artificial sequence
  Primer HuJH3-XhoI
gagtcattctcgactcgagacggtgaccattgtccc

SEQ ID NO: 110 - DNA, 36 bp, Artificial sequence
  Primer HuJH4/5-XhoI
gagtcattctcgactcgagacggtgaccagggttcc

SEQ ID NO: 111 - DNA, 36 bp, Artificial sequence
  Primer HuJH6-XhoI
gagtcattctcgactcgagacggtgaccgtggtccc

(19) United States
(12) Patent Application Publication
(10) Pub. No.: US 2009/0130652 A1
(43) Pub. Date: May 21, 2009

(54) Optimization of West Nile Virus Antibodies

(75) Inventors: Mark Throsby, Utrecht (NL); Cornelis Adriaan De Kruif, De Bilt (NL)

Correspondence Address: TRASKBRITT, P.C., P.O. BOX 2550, SALT LAKE CITY, UT, 84110, US

(73) Assignee: CRUCELL HOLLAND B.V., Leiden (NL)

(21) Appl. No.: 11/922,405

(22) PCT Filed: Jun. 22, 2006
(86) PCT No.: PCT/EP2006/063463

§ 371 Date: Dec. 13, 2007

Related U.S. Application Data
(60) Provisional application No. 60/693,365, filed on Jun. 23, 2005.

(30) Foreign Application Priority Data
Jun. 23, 2005 (EP) 05105625.7

(51) Int. Cl. C12Q 1/70; G01N 33/567

(52) U.S. Cl. 435/5; 435/7.21

(57) ABSTRACT

The invention relates to the production of binding molecules. In particular, the invention relates to methods for producing binding molecules having an improved functionality of interest.

### FIELD OF THE INVENTION

The invention relates to the production of binding molecules. In particular the invention relates to the production of binding molecules having an improved functionality of interest.

### BACKGROUND OF THE INVENTION

Traditionally, monoclonal antibodies have been prepared by the hybridoma technology. Over the last decade however, a variety of recombinant techniques have been developed that have revolutionized the generation of monoclonal antibodies and their engineering. Particularly, the development of antibody libraries and display technologies, such as phage display or more recently developed display technologies such as ribosome, yeast and bacterial display, have greatly influenced monoclonal antibody preparation.

In general, the established generation of antibody libraries in phages includes the cloning of repertoires of V genes (e.g., amplified from lymphocytes, plasma cells, hybridomas or any other immunoglobulin expressing population of cells or assembled in vitro) for display of associated heavy and light chain variable domains on the surface of the phages. Large repertoires of antibody clones with a potential diversity in excess of 10¹⁰ can be generated this way. From these repertoires selection for binding to a specific antigen can be performed thereby generating sub-libraries which can be used to generate antibodies. The antibodies can be expressed from bacteria infected with the phages and, optionally, the obtained antibodies can be further improved by means of suitable mutagenesis techniques.

A problem associated with the above described technique is the separate isolation of the variable region encoding sequences from the population of antibody producing cells. As a consequence thereof, during combinatorial library construction, heavy chain variable region and light chain variable regions from the antibodies originally present in the donor will be recombined randomly, resulting in the loss of the original pairing. The chances of recovering the exact heavy chain variable region and light chain variable region pairs as present in the donor from a combinatorial library are very limited. Even when a considerable amount of screening is performed, the achieved diversity of the repertoire might not be sufficiently large to isolate variable region encoding sequence pairs giving rise to antibodies of similar high functionality as those found in the original cells. Further, the enrichment procedures normally used to screen combinatorial libraries introduce a strong bias e.g. for polypeptides of particular low toxicity in E. coli, efficient folding, slow off-rates, or other system dependent parameters, that reduce the diversity of the library even further and therefore further decrease the chances of finding antibodies having the desired functionalities.

A known method for recovering original pairs of V genes and optimizing functionalities is chain shuffling (see Clackson et al. (1991) and Marks et al. (1992)). In this approach one of the two variable regions is fixed and combined with a repertoire of naturally occurring complementary variable regions to yield a secondary library. The thus obtained new combinations can be displayed on phages and searched for pairings having the desired functionality in terms of binding. A disadvantage of the chain shuffling method is that, although antibody fragment phage libraries are valuable tools for isolating antibodies with desired binding properties, the antibody phage format is not considered a suitable format in assays of functionalities other than binding, e.g. assays for testing neutralizing activity where bivalent or multivalent binding is required. In this instance, the neutralizing activity measured for phage antibodies (and even for scFv or Fab fragments derived from phage antibodies) is not representative of the neutralizing activity of the complete immunoglobulin molecules.

In view thereof, it would be desirable to have a technique for recovering original pairs of V gene heavy and light chains having the desired functionalities in all, or at least more aspects than binding alone.

The present invention provides a solution to the stated problem. The method combines a heavy chain gene from a selected functional first immunoglobulin molecule with a panel of light chain genes from second immunoglobulin molecules resulting in panels of immunoglobulin heavy and light chain expressing vectors that lead to specific immunoglobulin molecules. This way the original light chain gene or at least a light chain gene that better complements the heavy chain gene partner may be found. The ability to test the resulting heavy and light chain combinations directly as complete immunoglobulins is a major advantage as complete immunoglobulins can be used in many functionality assays.

The recombinant expression of the heavy chain of a first immunoglobulin molecule with the light chain of a second immunoglobulin molecule resulting in an immunoglobulin having heavy and light chain of different origin has been suggested in U.S. Pat. No. 6,331,415. However, in U.S. Pat. No. 6,331,415 the immunoglobulins combining heavy and light chains from different sources do not retain specificity for the antigen and are therefore said to lack in antibody character. For example in column 6, line 30 the person skilled in the art is warned that such “composite” immunoglobulins are non-specific immunoglobulins, i.e. an immunoglobulin lacking specificity for the antigen of choice. Based on the prior art a person skilled in the art would therefore have had no incentive, and would even have been hesitant, to express heavy and light chain of different immunoglobulin molecules for the preparation of immunoglobulin molecules having desired functionalities other than binding per se.

### DESCRIPTION OF THE FIGURES

FIG. 1 shows the ELISA binding of dilutions of the optimized antibodies CR4354L4261, CR4354L4267, CR4354L4328, CR4354L4335, CR4354L4383 and the parent antibody CR4354 to WNV. On the Y-axis the absorbance (OD) at 492 nm is shown and on the X-axis the amount of antibody in μg/ml is shown.

### SUMMARY OF THE INVENTION

The invention provides methods for obtaining immunoglobulin molecules having an improved functionality of interest. In a preferred embodiment, the method comprises combining the heavy chain variable gene of an immunoglobulin molecule having a desired functionality of interest with a panel of light chain variable genes resulting in panels of immunoglobulin heavy and light chain expressing vectors and selecting for immunoglobulin molecules having an improved functionality of interest.

### DETAILED DESCRIPTION OF THE INVENTION

In a first aspect the present invention encompasses a method of obtaining a binding molecule, e.g. an immunoglobulin molecule, with specificity for a pre-selected antigen having an improved functionality of interest, wherein the functionality of interest is other than binding specificity, said method comprising the steps of a) isolating a nucleic acid molecule encoding the heavy chain of a first immunoglobulin molecule, said first immunoglobulin molecule having specificity for the pre-selected antigen and a functionality of interest, b) transfecting a host with the nucleic acid molecule encoding the heavy chain of the first immunoglobulin molecule and a nucleic acid molecule encoding the light chain of a second immunoglobulin molecule, c) culturing the host under conditions conducive to the expression of a third immunoglobulin molecule, said third immunoglobulin molecule comprising the heavy chain of the first immunoglobulin molecule and the light chain of the second immunoglobulin molecule, d) determining whether the third immunoglobulin molecule still has specificity for the pre-selected antigen and e) determining the functionality of interest of the third immunoglobulin molecule and comparing it with the functionality of interest of the first immunoglobulin molecule, wherein steps d and e can be in either order or simultaneously, and f) selecting a third immunoglobulin molecule having an improved functionality of interest and still having specificity for the pre-selected antigen. The term “nucleic acid molecule” as used in the present invention refers to a polymeric form of nucleotides and includes both sense and antisense strands of RNA, cDNA, genomic DNA, and synthetic forms and mixed polymers of the above. A nucleotide refers to a ribonucleotide, deoxynucleotide or a modified form of either type of nucleotide. The term also includes single- and double-stranded forms of DNA. In addition, a polynucleotide may include either or both naturally-occurring and modified nucleotides linked together by naturally-occurring and/or non-naturally occurring nucleotide linkages. The nucleic acid molecules may be modified chemically or biochemically or may contain non-natural or derivatized nucleotide bases, as will be readily appreciated by those of skill in the art. The above term is also intended to include any topological conformation, including single-stranded, double-stranded, partially duplexed, triplex, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. A reference to a nucleic acid sequence encompasses its complement unless otherwise specified. Thus, a reference to a nucleic acid molecule having a particular sequence should be understood to encompass its complementary strand, with its complementary sequence.

In another embodiment only a nucleic acid molecule encoding the heavy chain variable region of a first immunoglobulin molecule is isolated. The nucleic acid molecule encoding the heavy chain variable region is then operably linked to a nucleic acid molecule encoding a heavy chain constant region and cloned into a vector, preferably an expression vector. This vector is subsequently used for transfecting a host according to step b of the method of obtaining an immunoglobulin molecule according to the invention. The heavy chain constant region encoding nucleic acid molecule which is operably linked to the nucleic acid molecule encoding the heavy chain variable region of the first immunoglobulin molecule can be identical to the heavy chain constant region encoding nucleic acid molecule originally found in the first immunoglobulin molecule, but alternatively it can also differ from the one found in the first immunoglobulin molecule. It can differ in such a way that it still provides a heavy chain constant region amino acid sequence identical to that translated from the nucleic acid molecule encoding the heavy chain constant region originally found in the first immunoglobulin molecule. Otherwise, the difference between the heavy chain constant region encoding nucleic acid molecules can be such that the heavy chain constant region comprises amino acid mutations (deletions, substitutions and/or insertions) compared to the heavy chain constant region of the first immunoglobulin molecule or, even stronger, the heavy chain constant region belongs to a different isotype or class than the heavy chain constant region of the first immunoglobulin molecule. It can thus be used to switch immunoglobulin classes or subclasses. If the nucleic acid molecule encoding the heavy chain or variable region thereof of a first immunoglobulin molecule has already been isolated or can be produced without actual isolation, e.g. synthetically based on sequence information, step a of the method may be redundant. Therefore, the present invention also contemplates the above method lacking step a. The starting point remains however a first immunoglobulin molecule with specificity for a pre-selected antigen and having a functionality of interest, wherein the functionality of interest is other than binding specificity.

The nucleic acid molecule encoding the light chain may be completely derived from or isolated from a single existing immunoglobulin molecule or may be produced by operably linking the nucleic acid molecule encoding the light chain variable region from one immunoglobulin molecule to a nucleic acid molecule encoding a light chain constant region of another immunoglobulin molecule. The nucleic acid molecule encoding the light chain is cloned into a vector, preferably an expression vector, which is subsequently used for transfecting a host according to step b of the method of the invention.

The nucleic acid molecule encoding the heavy chain of the first immunoglobulin molecule and the nucleic acid molecule encoding the light chain of the second immunoglobulin molecule may be expressed from separate expression vectors or may be expressed from a single expression vector. Vectors, i.e. nucleic acid constructs, comprising one or more nucleic acid molecules encoding immunoglobulin heavy and/or light chains are also covered by the present invention. Vectors can be used for cloning and/or for expression of the immunoglobulin heavy and/or light chains. The one or more nucleic acid molecules may be operably linked to one or more expression-regulating nucleic acid molecules. The term “expression-regulating nucleic acid sequence” as used herein refers to polynucleotide sequences necessary for and/or affecting the expression of an operably linked coding sequence in a particular host organism. The expression-regulating nucleic acid sequences, such as inter alia appropriate transcription initiation, termination, promoter, enhancer sequences; repressor or activator sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion, can be any nucleic acid sequence showing activity in the host organism of choice and can be derived from genes encoding proteins, which are either homologous or heterologous to the host organism. The identification and employment of expression-regulating sequences is routine to the person skilled in the art. The choice of the vectors is dependent on the recombinant procedures followed and the host used. Introduction of vectors in host cells can be effected by inter alia calcium phosphate transfection, virus infection, DEAE-dextran mediated transfection, lipofectamine transfection or electroporation. Vectors may be autonomously replicating or may replicate together with the chromosome into which they have been integrated. Preferably, the vectors contain one or more selection markers. The choice of the markers may depend on the host cells of choice, although this is not critical to the invention as is well known to persons skilled in the art. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the immunoglobulin heavy and/or light chains as described above operably linked to one or more nucleic acid molecules encoding proteins or peptides that can be used to isolate the immunoglobulin molecules are also covered by the invention. These proteins or peptides include, but are not limited to, glutathione-S-transferase, maltose binding protein, metal-binding polyhistidine, green fluorescent protein, luciferase and beta-galactosidase.

Hosts containing one or more copies of the vectors mentioned above are an additional subject of the present invention. Preferably, the hosts are host cells. Host cells include, but are not limited to, cells of mammalian, plant, insect, fungal or bacterial origin. Bacterial cells include, but are not limited to, cells from Gram positive bacteria such as several species of the genera Bacillus, Streptomyces and Staphylococcus or cells of Gram negative bacteria such as several species of the genera Escherichia, such as E. coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells such as Chinese Hamster Ovary (CHO) cells, COS cells, BHK cells or Bowes melanoma cells are preferred in the present invention. Mammalian cells provide expressed proteins with posttranslational modifications that are most similar to natural molecules of mammalian origin. Since the present invention deals with molecules that may have to be administered to humans, a completely human expression system would be particularly preferred. Therefore, even more preferably, the host cells are human cells. Examples of human cells are inter alia HeLa, 911, AT1080, A549, 293 and HEK293T cells. In preferred embodiments, the human producer cells comprise at least a functional part of a nucleic acid sequence encoding an adenovirus E1 region in expressible format. In even more preferred embodiments, said host cells are derived from a human retina and immortalised with nucleic acids comprising adenoviral E1 sequences, such as 911 cells or the cell line deposited at the European Collection of Cell Cultures (ECACC), CAMR, Salisbury, Wiltshire SP4 OJG, Great Britain on 29 Feb. 1996 under number 96022940 and marketed under the trademark PER.C6® (PER.C6 is a registered trademark of Crucell Holland B.V.). For the purposes of this application “PER.C6” refers to cells deposited under number 96022940 or ancestors, passages up-stream or downstream as well as descendants from ancestors of deposited cells, as well as derivatives of any of the foregoing. Production of recombinant proteins in host cells can be performed according to methods well known in the art. The use of the cells marketed under the trademark PER.C6® as a production platform for proteins of interest has been described in WO 00/63403 the disclosure of which is incorporated herein by reference in its entirety.

The hosts, preferably host cells, comprising the nucleic acid molecule encoding the heavy chain of the first immunoglobulin molecule and the nucleic acid molecule encoding the light chain of the second immunoglobulin molecule are cultured under conditions conducive to expression of a third immunoglobulin molecule, wherein said third immunoglobulin molecule comprises the heavy chain (or at least the variable region thereof) of the first immunoglobulin molecule and the light chain (or at least the variable region thereof) of the second immunoglobulin molecule. In an embodiment several hosts each expressing a different heavy chain-light chain combination, i.e. a different third immunoglobulin molecule, are cultured. The heavy chains (or at least the variable regions thereof) of each third immunoglobulin molecule are identical. Optionally, the expressed third immunoglobulin molecules are recovered. They can be recovered from the cell free extract, but preferably they are recovered from the culture medium. Methods to recover proteins, such as immunoglobulin molecules, from cell free extracts or culture medium are well known to the man skilled in the art.

Alternatively, next to the expression in hosts the third immunoglobulin molecules can be produced synthetically by conventional peptide synthesizers or in cell-free translation systems using the nucleic acid molecules encoding the immunoglobulin heavy and light chains.

Next, it is determined whether the expressed third immunoglobulin molecules still have specificity for the pre-selected antigen. This can be done by assays suitable for measuring the specificity of immunoglobulin molecules for pre-selected antigens which are known to the person skilled in the art. Moreover, the functionality of interest of the panel of third immunoglobulin molecules is determined, e.g. by means of an assay suitable for detecting and/or quantitating the functionality of interest, and the functionality of interest of each of the third immunoglobulin molecules produced is compared to the functionality of interest of the first immunoglobulin molecule. The step of determining whether the expressed third immunoglobulin molecules still have specificity for the pre-selected antigen and the step of determining the functionality of interest of the panel of third immunoglobulin molecules and comparing it to the functionality of interest of the first immunoglobulin molecule can be in either order or performed simultaneously. Subsequently, third immunoglobulin molecules having an improved functionality of interest and still having specificity for the pre-selected antigen are isolated. The functionality of interest of the third immunoglobulin molecules isolated is improved compared to the functionality of interest of the first immunoglobulin molecule. In a preferred embodiment improved functionality of interest as used herein means an increased, higher or enhanced functionality of interest.

Preferably, the pre-selected antigen is from an organism selected from the group consisting of a virus, a protozoa, a bacterium, a yeast, a fungus and a parasite. Pre-selected antigens include, but are not limited to, the complete virus, protozoa, bacterium, yeast, fungus or parasite, either in active form or inactivated or attenuated, as well as parts thereof including inter alia (glyco)proteins, (poly)peptides, (poly)saccharides, carbohydrates, (glyco)lipids, phospholipids, lipopolysaccharides, peptidoglycans, (lipo)teichoic acids, other antigenic molecules, and parts, fragments, and derivatives thereof. It is to be understood that specificity for a pre-selected antigen does not exclude binding to a different epitope on the same antigen. In a preferred embodiment the functionality of interest is selected from the group consisting of affinity for the pre-selected antigen, neutralizing activity, opsonic activity, or any other biological activity, e.g. complement fixing activity or recruitment and attachment of immune effector cells such as neutrophils, macrophages, NK cells, etc. Both of the latter activities require the presence of a glycosylated Fc portion of the immunoglobulin molecule. These activities may also be enhanced when the immunoglobulin is able to interact in a bivalent or multivalent fashion. Additionally, intrinsic activities toward infectious agents frequently require cross-linking of surface molecules. E.g. optimal bactericidal and bacterial static activities against bacteria or neutralizing activity against viruses may only be measured when the immunoglobulin is able to interact in a bivalent or multivalent fashion. Full immunoglobulins also allow the measurement of a protective effect in vivo that may or may not be predicted from in vitro assays. These protective effects frequently may involve complex interactions of immunological effector cells with the Fc portion of the antibody as well as interaction with infectious organisms that require multivalent attachment such as immune complex formation and clearance. Assays for detecting the functionalities are well known to the person skilled in the art and include, but are not limited to, CDC, ADCC, opsonisation assays, phagocytic assays, complement fixing assays, growth inhibition assays, neutralization of infectivity, internalization assays (see e.g. Coligan J E, Kruisbeek A M, Margulies D H, Shevach E M and Strober W (eds), 1991, Current Protocols in Immunology, 1-2, Greene Publishing Associates and Whiley-Interscience, New York; Robinson J P and Babcock G F (eds), 1998, Phagocytic Function: A guide for research and clinical evaluation, Wiley-Liss, New York; Weir D M et al. (eds), Handbook of Experimental Immunology, volume 4, fifth edition, Blackwell Scientific Publications, Oxford; Collins and Lyne's Microbiological Methods, 1994, Seventh edition, CH Collins, Butterworth-Heinemann).

In a preferred embodiment the light chain of the first immunoglobulin molecule and the light chain of the second immunoglobulin molecule are members of the same gene family and even more preferably the light chain of the first immunoglobulin molecule and the light chain of the second immunoglobulin molecule are members of the same germline. In another embodiment of the invention the heavy chain of the first immunoglobulin molecule and the heavy chain of the second immunoglobulin molecule are members of the same gene family. The heavy chain of the first immunoglobulin molecule and the heavy chain of the second immunoglobulin molecule might even belong to the same germline. Preferably, the heavy chain CDR3 regions of the first and second immunoglobulin molecules are similar or even identical. Heavy chain variable region genes can accommodate a range of light chain variable region genes to form a functional binding site. In inter alia phage display, heavy chain variable regions are selected that have paired with light chain variable regions that “fit”, meaning that the variable heavy chain/variable light chain pair is functional, resulting in e.g. binding to an antigen of interest. Although many different light chain variable regions may pair with a given heavy chain variable region to form functional immunoglobulin molecules, these light chain variable regions usually lack the exact complementary somatic mutations as introduced during affinity maturation, rendering a functionality of interest such as affinity for a pre-selected antigen or neutralizing activity suboptimal. It has now been found that within a panel of immunoglobulin molecules all having specificity for a pre-selected antigen, the light chain variable region genes can be grouped in panels based on their similarity to a light chain variable region gene family or germline family, resulting in groups of closely related light chain variable region genes. The light chain gene of the antibody having the desired functionality can be grouped in one of these panels. By combining the selected functional heavy chain variable region gene with the complete set of light chain genes in this panel, it may be possible to identify the original light chain variable region gene or at least light chain variable region genes that better complement the heavy chain variable region partner resulting in a panel of immunoglobulins having an improved functionality of interest and still having specificity for the pre-selected antigen.

Another aspect of the invention includes a first immunoglobulin molecule, which is obtained or derived from a collection of binding molecules displayed on the surface of replicable genetic display packages. Typically, the collection of binding molecules is contacted with a target of interest under conditions conducive to binding. Next, at least once is selected for a replicable genetic package binding to the target of interest and a replicable genetic package binding to the target of interest is isolated and recovered from replicable genetic packages that do not bind to the target of interest. Finally, the binding molecule and/or the nucleic acid molecule encoding the binding molecule is isolated from the recovered replicable genetic package and combined with standard molecular biological techniques to make constructs encoding inter alia complete immunoglobulin molecules. These constructs can be transfected into suitable cell lines and complete immunoglobulin molecules (e.g. IgG, IgA or IgM) can be produced (see Huls et al., 1999; Boel et al., 2000). The produced immunoglobulin molecules can be tested for specificity for the pre-selected antigen and for the desired functionality other than binding specificity. The pre-selected antigen may be identical or essentially similar to the target of interest, but may also be derived from the target of interest, e.g. in case the target of interest is an infectious agent and the pre-selected antigen is a polypeptide of the infectious agent. In an embodiment the first and second immunoglobulin molecules are both form one or more pools of immunoglobulin molecules selected against the pre-selected antigen.

The replicable genetic package is preferably selected from the group consisting of (bacterio)phages, bacteria, yeasts, fungi, viruses, and a spore of a microorganism. Most preferably, the replicable genetic package is a (bacterio)phage. Alternatively, the first immunoglobulin molecule can be obtained from a collection of binding molecules displayed by means of e.g. ribosome display, mRNA display, CIS display. In a preferred embodiment, the first immunoglobulin molecule and the second immunoglobulin molecule are both obtained from the same collection of binding molecules. Preferably, all second immunoglobulin molecules used are obtained from the same collection of binding molecules. The binding molecules are preferably displayed, i.e. they are attached to a group or molecule located at an exterior surface of the replicable genetic package, on the replicable genetic packages in the format of scFv or Fab fragments. However, suitable binding molecules include any (poly)peptides that contain at least a fragment of an immunoglobulin that is sufficient to confer specific antigen binding to the (poly)peptide. Binding molecules may have more than one binding site and may have more than one antigen specificity. Generally, the replicable genetic package is a screenable unit comprising a binding molecule to be screened linked to a nucleic acid molecule encoding the binding molecule. The nucleic acid molecule should be replicable either in vivo (e.g., as a vector) or in vitro (e.g., by PCR, transcription and translation). In vivo replication can be autonomous (as for a cell), with the assistance of host factors (as for a virus) or with the assistance of both host and helper virus (as for a phagemid). Replicable genetic packages displaying a collection of binding molecules are formed by introducing nucleic acid molecules encoding exogenous binding molecules to be displayed into the genomes of the replicable genetic packages to form fusion proteins with endogenous proteins that are normally expressed from the outer surface of the replicable genetic packages. Expression of the fusion proteins, transport to the outer surface and assembly results in display of exogenous binding molecules from the outer surface of the replicable genetic packages. As mentioned before the preferred replicable genetic package is a phage. Phage display methods for identifying and obtaining immunoglobulin molecules, e.g. monoclonal antibodies, are by now well-established methods known by the person skilled in the art. They are e.g. described in U.S. Pat. No. 5,696,108; Burton and Barbas, 1994; de Kruif et al., 1995b; and Phage Display: A Laboratory Manual. Edited by: CF Barbas, D R Burton, J K Scott and G J Silverman (2001), Cold Spring Harbor Laboratory Press, Cold Spring Harbor, N.Y. All these references are herewith incorporated herein in their entirety. For the construction of phage display libraries, collections of heavy and light chain variable region genes from e.g. IgG or IgM are expressed on the surface of (bacterio)phage particles, preferably filamentous bacteriophages, in for example single-chain Fv (scFv) or in Fab format (see de Kruif et al., 1995b). Large libraries of antibody fragment-expressing phages typically contain more than 1.0*10⁹ antibody specificities and may be assembled from the immunoglobulin V regions expressed in the B lymphocytes of immunized or non-immunized individuals. In a specific embodiment of the invention the phage library of binding molecules, preferably scFv phage library, is prepared from RNA isolated from cells obtained from a subject that has been vaccinated or exposed to an infectious agent. The infectious agent can be selected form the group consisting of a virus, a protozoa, a bacterium, a yeast, a fungus or a parasite. RNA can be isolated from inter alia bone marrow or peripheral blood, preferably peripheral blood lymphocytes. The subject can be an animal vaccinated or exposed to an infectious agent, but is preferably a human subject that has been vaccinated or has been knowingly or unknowingly exposed to an infectious agent. Preferably the human subject has recovered from an infectious agent. Preferably, the cells from which the RNA is isolated are obtained from one subject. Phage display libraries may also be constructed from immunoglobulin variable regions that have been partially assembled in vitro to introduce additional antibody diversity in the library (semi-synthetic libraries). For example, in vitro assembled variable regions contain stretches of synthetically produced, randomized or partially randomized DNA in those regions of the molecules that are important for antibody specificity, e.g. CDR regions. Phage display libraries comprising completely synthetic immunoglobulin variable regions may also be used. Specific phage antibodies can be selected from the library by for instance immobilising target antigens, purified or produced recombinantly, on a solid phase and subsequently exposing the target antigens to a phage library to allow binding of phages expressing binding molecules fragments specific for the solid phase-bound antigen(s). Non-bound phages are removed by washing and bound phages eluted from the solid phase for infection of E. coli bacteria and subsequent propagation. Multiple rounds of selection and propagation are usually required to sufficiently enrich for phages binding specifically to the target antigen(s). Phages may also be selected for binding to complex antigens such as complex mixtures of proteins or (poly)peptides of interest, fusion protein comprising proteins or (poly)peptides of interest, host cells expressing one or more proteins or (poly)peptides of interest, virus-like particles comprising proteins of interest, whole (activated or inactivated) infectious agents such as viruses, bacteria, parasites, fungi, yeasts, etc, or any of the pre-selected antigens mentioned before and parts, fragments or derivatives thereof. Extracellularly exposed parts of molecules from infectious agents can also be used as selection material. The selection material used can be immobilised or non-immobilised. In a specific embodiment the selection can be performed on different materials derived from the infectious agents. For instance, the first selection round can be performed on an activated or inactivated infectious agent, while the second and third selection round can be performed on a protein from the infectious agent and virus-like particles from the infectious agent, respectively. Of course, other combinations are also suitable. Different materials can also be used during one selection/panning step. If necessary, the infectious agents can be inactivated before selection takes place. Methods for inactivating/attenuating e.g. viruses or bacteria are well known in the art and include, but are not limited to, treatment with specific chemicals, heat inactivation, inactivation by UV irradiation, inactivation by gamma irradiation. The viruses or bacteria may be isolated before or after inactivation. Purification where necessary may be performed by means of well-known purification methods suitable for viruses or bacteria such as for instance centrifugation through a glycerol cushion or centrifugation. Methods to test if a virus or bacterium is still infective/viable or partly or completely inactivated are also well-known to the person skilled in the art.

If desired, before exposing the phage library to target antigens the phage library can first be subtracted by exposing the phage library to e.g. non-target antigens or host cells comprising no target molecules or non-target molecules that are similar, but not identical, to the target, and thereby strongly enhance the chance of finding relevant binding molecules (This process is referred to as the MAbstract® process. MAbstract® is a registered trademark of Crucell Holland B.V., see also U.S. Pat. No. 6,265,150 which is incorporated herein by reference).

As used herein, “virus-like particle” refers to a virus particle that assembles into intact enveloped viral structures. A virus-like particle does however not contain genetic information sufficient to replicate. Virus-like particles have essentially a similar physical appearance as the wild-type virus, i.e. they are morphologically and antigenically essentially similar to authentic virions. The virus-like particles as used herein may comprise wild-type viral amino acid sequences. The virus-like particles may also include functional copies of certain genes. Furthermore, the virus-like particles may also include foreign nucleic acid. The virus-like particles can be naturally or non-naturally occurring viral particles. They may lack functional copies of certain genes of the wild-type virus, and this may result in the virus-like particle being incapable of some function which is characteristic of the wild-type virus, such as replication and/or cell-cell movement. The missing functional copies of the genes can be provided by the genome of a host cell or on a plasmid present in the host cell, thereby restoring the function of the wild-type virus to the virus-like particle when in the host cell. Preferably, virus-like particles display the same cellular tropism as the wild-type virus. The virus-like particle may be non-infectious, but is preferably infectious. The term “infectious” as used herein means the capacity of the virus-like particle to complete the initial steps of viral cycle that lead to cell entry. In an embodiment of the above methods of the invention the virus-like particle self assembles. In another embodiment the above methods are performed using pseudoviruses instead of virus-like particles. Pseudoviruses and their production are well known to the skilled person. Preferably, the pseudoviruses as used herein comprise a heterologous viral envelope protein on their surface. Virus-like particles can be produced in suitable host cells such as inter alia mammalian cells as described above. They can be produced intracellularly and/or extracellularly and can be harvested, isolated and/or purified as intact virus-like particles by means known to the skilled person such as inter alia affinity chromatography, gel filtration chromatography, ion exchange chromatography, and/or density gradient sedimentation. The protein comprised in and/or on the virus-like particle can be a viral structural protein. Preferably, the protein is a protein present on the surface of the virus such as a viral envelope protein. The protein may be wild-type, modified, chimaeric, or a part thereof. Preferably, the virus-like particle is produced extracellularly when proteins are expressed in host cells, preferably human host cells.

In an embodiment of the invention the first immunoglobulin molecule and the second immunoglobulin molecule both have the functionality of interest, although not necessarily in amount. In another embodiment of the invention first and second immunoglobulin molecules have specificity for the pre-selected antigen.

In a preferred embodiment the first, second and third immunoglobulin molecule are human, however immunoglobulin molecules of other species, or chimeric or humanized immunoglobulin molecules may also be used. The term “human”, when applied to immunoglobulin molecules, refers to molecules that are either directly derived from a human or based upon a human sequence. When an immunoglobulin molecule is derived from or based on a human sequence and subsequently modified, it is still to be considered human as used throughout the specification. In other words, the term human, when applied to immunoglobulin molecules is intended to include immunoglobulin molecules having variable and constant regions derived from human germline immunoglobulin sequences, based on variable or constant regions either or not occurring in a human or human lymphocyte or in modified form. Thus, the human immunoglobulin molecules may include amino acid residues not encoded by human germline immunoglobulin sequences, comprise substitutions and/or deletions (e.g., mutations introduced by for instance random or site-specific mutagenesis in vitro or by somatic mutation in vivo). “Based on” as used herein refers to the situation that a nucleic acid sequence may be exactly copied from a template, or with minor mutations, such as by error-prone PCR methods, or synthetically made matching the template exactly or with minor modifications. Semisynthetic molecules based on human sequences are also considered to be human as used herein. The term “immunoglobulin molecule” includes all immunoglobulin classes and subclasses known in the art. Depending on the amino acid sequence of the constant domain of their heavy chains, binding molecules can be divided into the five major classes of intact antibodies: IgA, IgD, IgE, IgG, and IgM, and several of these may be further divided into subclasses (isotypes), e.g., IgA1, IgA2, IgG1, IgG2, IgG3 and IgG4. Preferably, immunoglobulin molecules are selected from the group consisting of IgA, IgD, IgE, IgG, and IgM. In a specific embodiment the immunoglobulin molecules are monoclonal antibodies, preferably human monoclonal antibodies.

An immunoglobulin molecule obtainable by the methods according to the invention is another aspect of the invention. Also a nucleic acid molecule encoding such an immunoglobulin molecule is part of the present invention, as well as a pharmaceutical composition comprising at least an immunoglobulin molecule obtainable by a method according to the invention and at least one pharmaceutically acceptable excipient. Pharmaceutical compositions may further comprise other molecules suitable for the prophylaxis and/or treatment of an infectious agent.

### EXAMPLES

To illustrate the invention, the following examples are provided. The examples are not intended to limit the scope of the invention in any way.

### Example 1

### Construction of a ScFv Phage Display Library Using RNA Extracted from Peripheral Blood of WNV Convalescent Donors

From three convalescent WNV patients samples of blood were taken 1, 2 and 3 months after infection. Peripheral blood leukocytes were isolated by centrifugation and the blood serum was saved and frozen at −80° C. All donors at all time points had high titres of neutralising antibodies to WNV as determined using a virus neutralisation assay. Total RNA was prepared from the cells using organic phase separation and subsequent ethanol precipitation. The obtained RNA was dissolved in RNAse free water and the concentration was determined by OD260 nm measurement. Thereafter, the RNA was diluted to a concentration of 100 ng/μl. Next, 1 μg of RNA was converted into cDNA as follows: To 10 μl total RNA, 13 μl DEPC-treated ultrapure water and 1 μl random hexamers (500 ng/μl) were added and the obtained mixture was heated at 65° C. for 5 minutes and quickly cooled on wet-ice. Then, 8 μl 5× First-Strand buffer, 2 μl dNTP (10 mM each), 2 μl DTT (0.1 M), 2 μl Rnase-inhibitor (40 U/μl) and 2 μl Superscript™III MMLV reverse transcriptase (200 U/μl) were added to the mixture, incubated at room temperature for 5 minutes and incubated for 1 hour at 50° C. The reaction was terminated by heat inactivation, i.e. by incubating the mixture for 15 minutes at 75° C.

The obtained cDNA products were diluted to a final volume of 200 μl with DEPC-treated ultrapure water. The OD260 nm of a 50 times diluted solution (in 10 mM Tris buffer) of the dilution of the obtained cDNA products gave a value of 0.1.

For each donor 5 to 10 μl of the diluted cDNA products were used as template for PCR amplification of the immunoglobulin gamma heavy chain family and kappa or lambda light chain sequences using specific oligonucleotide primers (see Tables 1-6). PCR reaction mixtures contained, besides the diluted cDNA products, 25 pmol sense primer and 25 pmol anti-sense primer in a final volume of 50 μl of 20 mM Tris-HCl (pH 8.4), 50 mM KCl, 2.5 mM MgCl2, 250 μM dNTPs and 1.25 units Taq polymerase. In a heated-lid thermal cycler having a temperature of 96° C., the mixtures obtained were quickly melted for 2 minutes, followed by 30 cycles of: 30 seconds at 96° C., 30 seconds at 60° C. and 60 seconds at 72° C.

In a first round amplification, each of seventeen light chain variable region sense primers (eleven for the lambda light chain (see Table 1) and six for the kappa light chain (see Table 2)) were combined with an anti-sense primer recognizing the C-kappa called HuCk 5′-ACACTCTCCCCTGTTGAAGCT CTT-3′ (SEQ ID NO:1) or C-lambda constant region HuCλ2 5′-TGAACATTCTGTAGGGGCCACTG-3′ (SEQ ID NO:2) and HuCλ7 5′-AGAGCATTCTGCAGGGGCCACTG-3′ (SEQ ID NO:3) (the HuCλ2 and HuCλ7 anti-sense primers were mixed to equimolarity before use), yielding 4 times 17 products of about 600 basepairs. These products were purified on a 2% agarose gel and isolated from the gel using Qiagen gel-extraction columns. 1/10 of each of the isolated products was used in an identical PCR reaction as described above using the same seventeen sense primers, whereby each lambda light chain sense primer was combined with one of the three Jlambda-region specific anti-sense primers and each kappa light chain sense primer was combined with one of the five Jkappa-region specific anti-sense primers. The primers used in the second amplification were extended with restriction sites (see Table 3) to enable directed cloning in the phage display vector PDV-C06 (see SEQ ID NO:4). This resulted in 4 times 63 products of approximately 350 basepairs that were pooled to a total of 10 fractions. This number of fractions was chosen to maintain the natural distribution of the different light chain families within the library and not to over or under represent certain families. The number of alleles within a family was used to determine the percentage of representation within a library (see Table 4). In the next step, 2.5 μg of pooled fraction and 100 μg PDV-C06 vector were digested with SalI and NotI and purified from gel. Thereafter, a ligation was performed overnight at 16° C. as follows. To 500 ng PDV-C06 vector 70 ng pooled fraction was added in a total volume of 50 μl ligation mix containing 50 mM Tris-HCl (pH 7.5), 10 mM MgCl₂, 10 mM DTT, 1 mM ATP, 25 μg/ml BSA and 2.5 μl T4 DNA Ligase (400 U/μl). This procedure was followed for each pooled fraction. The ligation mixes were purified by phenol/chloroform, followed by a chloroform extraction and ethanol precipitation, methods well known to the skilled artisan. The DNA obtained was dissolved in 50 μl ultrapure water and per ligation mix two times 2.5 μl aliquots were electroporated into 40 μl of TG1 competent E. coli bacteria according to the manufacturer's protocol (Stratagene). Transformants were grown overnight at 37° C. in a total of 30 dishes (three dishes per pooled fraction; dimension of dish: 240 mm×240 mm) containing 2TY agar supplemented with 50 μg/ml ampicillin and 4.5% glucose. A (sub)library of variable light chain regions was obtained by scraping the transformants from the agar plates. This (sub)library was directly used for plasmid DNA preparation using a Qiagen™ QIAFilter MAXI prep kit.

For each donor the heavy chain immunoglobulin sequences were amplified from the same cDNA preparations in a similar two round PCR procedure and identical reaction parameters as described above for the light chain regions with the proviso that the primers depicted in Tables 5 and 6 were used. The first amplification was performed using a set of nine sense directed primers (see Table 5; covering all families of heavy chain variable regions) each combined with an IgG specific constant region anti-sense primer called HuCIgG 5′-GTC CAC CTT GGT GTT GCT GGG CTT-3′ (SEQ ID NO:5) yielding four times nine products of about 650 basepairs. These products were purified on a 2% agarose gel and isolated from the gel using Qiagen gel-extraction columns. 1/10 of each of the isolated products was used in an identical PCR reaction as described above using the same nine sense primers, whereby each heavy chain sense primer was combined with one of the four JH-region specific anti-sense primers. The primers used in the second round were extended with restriction sites (see Table 6) to enable directed cloning in the light chain (sub)library vector. This resulted per donor in 36 products of approximately 350 basepairs. These products were pooled for each donor per used (VH) sense primer into nine fractions. The products obtained were purified using Qiagen PCR Purification columns. Next, the fractions were digested with SfiI and XhoI and ligated in the light chain (sub)library vector, which was cut with the same restriction enzymes, using the same ligation procedure and volumes as described above for the light chain (sub)library. Alternatively, the fractions were digested with NcoI and XhoI and ligated in the light chain vector, which was cut with the same restriction enzymes, using the same ligation procedure and volumes as described above for the light chain (sub)library. Ligation purification and subsequent transformation of the resulting definitive library was also performed as described above for the light chain (sub)library and at this point the ligation mixes of each donor were combined per VH pool. The transformants were grown in 27 dishes (three dishes per pooled fraction; dimension of dish: 240 mm×240 mm) containing 2TY agar supplemented with 50 μg/ml ampicillin and 4.5% glucose. All bacteria were harvested in 2TY culture medium containing 50 μg/ml ampicillin and 4.5% glucose, mixed with glycerol to 15% (v/v) and frozen in 1.5 ml aliquots at −80° C. Rescue and selection of each library were performed as described below.

### Example 2

### Selection of Phages Carrying Single Chain Fv Fragments Specifically Recognizing WNV Envelope (E) Protein

Antibody fragments were selected using antibody phage display libraries, general phage display technology and MAbstract® technology, essentially as described in U.S. Pat. No. 6,265,150 and in WO 98/15833 (both of which are incorporated by reference herein). The antibody phage immune library was prepared as described in Example 1. Furthermore, the methods and helper phages as described in WO 02/103012 (incorporated by reference herein) were used in the present invention. For identifying phage antibodies recognizing WNV E protein, phage selection experiments were performed using whole WNV (called strain USA99b or strain 385-99) inactivated by gamma irradiation (50 Gy for 1 hour), recombinantly expressed WNV E protein (strain 382-99), and/or WNV-like particles expressing WNV E protein (strain 382-99) on their surface.

The recombinantly expressed E protein was produced as follows. The nucleotide sequence coding for the preM/M protein and the full length E protein of WNV strain 382-99 (see SEQ ID NO:6 for the amino acid sequence of a fusion protein comprising both WNV polypeptides) was synthesised. Amino acids 1-93 of SEQ ID NO:6 constitute the WNV preM protein, amino acids 94-168 of SEQ ID NO:6 constitute the WNV M protein, amino acids 169-669 of SEQ ID NO:6 constitute the WNV E protein (the soluble WNV E protein (ectodomain) constitutes amino acids 169-574 of SEQ ID NO:6, while the WNV E protein stem and transmembrane region constitutes amino acids 575-669 of SEQ ID NO:6) The synthesised nucleotide sequence was cloned into the plasmid pAdapt and the plasmid obtained was called pAdapt.WNV.prM-E (FL).

To produce a soluble secreted form of the E protein a construct was made lacking the transmembrane spanning regions present in the final 95 amino acids at the carboxyl terminal of the full length E protein (truncated form). For that purpose the full length construct pAdapt.WNV.prM-E (FL) was PCR amplified with the primers CMV-Spe (SEQ ID NO:7) and WNV-E-95 REV (SEQ ID NO:8) and the fragment obtained was cloned into the plasmid pAdapt.myc.his to create the plasmid called pAdapt.WNV-95. Next, the region coding for the preM protein, the truncated E protein, the Myc tag and His tag were PCR amplified with the primers clefsmaquwnv (SEQ ID NO:9) and reverse WNVmychis (SEQ ID NO:10) and cloned into the vector pSyn-C03 containing the HAVT20 leader peptide using the restriction sites EcoRI and SpeI. The expression construct obtained, pSyn-C03-WNV-E-95, was transfected into 90% confluent HEK293T cells using lipofectamine according to the manufacturers instructions. The cells were cultured for 5 days in serum-free ultra CHO medium, then the medium was harvested and purified by passage over HisTrap chelating columns (Amersham Bioscience) pre-charged with nickel ions. The truncated E protein was eluted with 5 ml of 250 mM imidazole and further purified by passage over a G-75 gel filtration column equilibrated with phosphate buffered saline (PBS). Fractions obtained were analysed by SDS-PAGE analysis and Western blotting using the WNV-E protein specific murine antibody 7H2 (Biorelience, see Beasley and Barrett 2002). Three 5 ml fractions containing a single band of ˜45 kDa that was immunoreactive with antibody 7H2 were aliquoted and stored at −20° C. until further use. The protein concentration was determined by OD280 nm.

WNV-like particles were produced as follows. The construct pSyn-C03-WNV-E-95 described above and pcDNA3.1 (Invitrogen) were digested with the restriction endonucleases MunI and XbaI and the construct pAdapt.WNV.prM-E (FL) described above was digested with the restriction endonucleases ClaI and XbaI. The resulting fragments were combined in a three point ligation to produce the construct pSyn-H-preM/E FL. This construct contained the full length E protein and expressed the two structural WNV proteins, protein M and E, required for assembly of an enveloped viron. The construct was transfected into 70% confluent HEK293T cells using lipofectamine according to the manufacturers instructions. The cells were cultured for 3 days in serum-free ultra CHO medium, then the medium was harvested, layered on to a 30% glycerol solution at a 2:1 ratio and pelleted by centrifugation for 2 h at 120,000*g at 4° C. The WNV-like particles were resuspended in PBS, aliquoted and stored at −80° C. Aliquots were analysed by SDS-PAGE analysis and Western blotting using the WNV-E protein specific murine antibody 7H2 (Biorelience).

Before inactivation, whole WNV was purified by pelleting through a 30% glycerol solution as described above for WNV-like particles. The purified WNV was resuspended in 10 mM Tris/HCl pH 7.4 containing 10 mM EDTA and 200 mM NaCl, the obtained preparation was kept on dry ice during inactivation, tested for infectivity and stored at −80° C. in small aliquots. Aliquots were analysed by SDS-PAGE analysis and Western blotting using the WNV-E protein specific murine antibody 7H2 (Biorelience).

Whole inactivated WNV, WNV-like particles or recombinantly expressed soluble E protein were diluted in PBS. 2-3 ml of the preparation was added to MaxiSorp™ Nunc-Immuno Tubes (Nunc) and incubated overnight at 4° C. on a rotating wheel. An aliquot of a phage library (500 μl, approximately 10¹³ cfu, amplified using CT helper phage (see WO 02/103012)) was blocked in blocking buffer (2% Protifar in PBS) for 1-2 hours at room temperature. The blocked phage library was added to the immunotubes, incubated for 2 hours at room temperature, and washed with wash buffer (0.1% v/v Tween-20 in PBS) to remove unbound phages. Bound phages were eluted from the antigen by incubation with 1 ml of 50 mM Glycine-HCl pH 2.2 for 10 minutes at room temperature. Subsequently, the eluted phages were mixed with 0.5 ml of 1 M Tris-HCl pH 7.5 to neutralize the pH. This mixture was used to infect 5 ml of a XL1-Blue E. coli culture that had been grown at 37° C. to an OD600 nm of approximately 0.3. The phages were allowed to infect the XL1-Blue bacteria for 30 minutes at 37° C. Then, the mixture was centrifuged for 10 minutes at 3200*g at room temperature and the bacterial pellet was resuspended in 0.5 ml 2-trypton yeast extract (2TY) medium. The obtained bacterial suspension was divided over two 2TY agar plates supplemented with tetracyclin, ampicillin and glucose. After incubation overnight of the plates at 37° C., the colonies were scraped from the plates and used to prepare an enriched phage library, essentially as described by De Kruif et al. (1995a) and WO 02/103012. Briefly, scraped bacteria were used to inoculate 2TY medium containing ampicillin, tetracycline and glucose and grown at a temperature of 37° C. to an OD600 nm of ˜0.3. CT helper phages were added and allowed to infect the bacteria after which the medium was changed to 2TY containing ampicillin, tetracycline and kanamycin. Incubation was continued overnight at 30° C. The next day, the bacteria were removed from the 2TY medium by centrifugation after which the phages in the medium were precipitated using polyethylene glycol (PEG) 6000/NaCl. Finally, the phages were dissolved in 2 ml of PBS with 1% bovine serum albumin (BSA), filter-sterilized and used for the next round of selection.

Typically, two rounds of selections were performed before isolation of individual phage antibodies. After the second round of selection, individual E. coli colonies were used to prepare monoclonal phage antibodies. Essentially, individual colonies were grown to log-phase in 96 well plate format and infected with CT helper phages after which phage antibody production was allowed to proceed overnight. The produced phage antibodies were PEG/NaCl-precipitated and filter-sterilized and tested in ELISA for binding to WNV-like particles purified as described supra.

### Example 3

### Validation of the WNV Specific Single-Chain Phage Antibodies

Selected single-chain phage antibodies that were obtained in the screens described above were validated in ELISA for specificity, i.e. binding to WNV E protein, whole inactivated WNV and WNV-like particles, all purified as described supra. Additionally, the single-chain phage antibodies were also tested for binding to 5% FBS. For this purpose, whole inactivated WNV, the WNV E protein, WNV-like particles or 5% FBS preparation was coated to Maxisorp™ ELISA plates. In addition whole inactivated rabies virus was coated onto the plates as a control. After coating, the plates were blocked in PBS containing 1% Protifar for 1 hour at room temperature. The selected single-chain phage antibodies were incubated for 15 minutes in an equal volume of PBS containing 1% Protifar to obtain blocked phage antibodies. The plates were emptied, and the blocked single-chain phage antibodies were added to the wells. Incubation was allowed to proceed for one hour, the plates were washed in PBS containing 0.1% v/v Tween-20 and bound phage antibodies were detected (using OD492 nm measurement) using an anti-M13 antibody conjugated to peroxidase. As a control, the procedure was performed simultaneously without single-chain phage antibody and a negative control single-chain phage antibody directed against rabies virus glycoprotein (antibody called SC02-447). 137 Single-chain phage antibodies that were specific for WNV were found (data not shown).

### Example 4

### Characterization of the WNV Specific ScFvs

From the selected specific single-chain phage antibody (scFv) clones plasmid DNA was obtained and nucleotide sequences were determined according to standard techniques (data not shown). The VH and VL gene identity (see Tomlinson I M, Williams S C, Ignatovitch O, Corbett S J, Winter G. V-BASE Sequence Directory. Cambridge United Kingdom: MRC Centre for Protein Engineering (1997)) of the scFvs specifically binding WNV are depicted in Table 7.

### Example 5

### Construction of Fully Human Immunoglobulin Molecules (Human Monoclonal Anti-WNV Antibodies) from the Selected Anti-WNV Single Chain Fvs

Heavy and light chain variable regions of the characterized scFvs were PCR-amplified using oligonucleotides to append restriction sites and/or sequences for expression in the IgG expression vectors pSyn-C18-HCγ1 (see SEQ ID NO:11), pSyn-C04-Clambda (see SEQ ID NO:12) and pSyn-C05-Ckappa (see SEQ ID NO:13). Alternatively, heavy and light chain variable regions of the characterized scFvs were cloned directly by restriction digest for expression in the IgG expression vectors pIg-C911-HCgamma1 (see SEQ ID No:14), pIg-C910-Clambda (see SEQ ID No:15) or pIG-C909-Ckappa (see SEQ ID NO:16). Nucleotide sequences for all constructs were verified according to standard techniques known to the skilled artisan. The resulting expression constructs encoding the anti-WNV human IgG1 heavy and light chains were transiently expressed in combination in 293T cells and supernatants containing human IgG1 antibodies were obtained and produced using standard purification procedures. The human anti-WNV IgG1 antibodies were validated for their ability to bind to irradiated WNV in ELISA essentially as described for scFvs (data not shown). Three dilutions of the respective antibodies in blocking buffer were tested. The positive control was the murine anti-WNV antibody 7H2 and the negative control was an anti-rabies virus antibody.

### Example 6

### In Vitro Neutralization of WNV by WNV Specific IgGs (Virus Neutralization Assay)

In order to determine whether the IgG1 molecules are capable of blocking WNV infection, in vitro virus neutralization assays (VNA) were performed. The VNA were performed on Vero cells (ATCC CCL 81). The WNV strain 385-99 which was used in the assay was diluted to a titer of 4×10³ TCID₅₀/ml (50% tissue culture infective dose per ml), with the titer calculated according to the method of Spearman and Kaerber. The IgG1 containing supernatants were serially 2-fold-diluted in PBS starting from 1:2 (1:2-1:1024). 25 μl of the respective scFv dilution was mixed with 25 μl of virus suspension (100 TCID₅₀/25 μl) and incubated for one hour at 37° C. The suspension was then pipetted twice in triplicate into 96-well plates. Next, 50 μl of a freshly trypsinized and homogenous suspension of Vero cells (1:3 split of the confluent cell monolayer of a T75-flask) resuspended in DMEM with 10% v/v fetal calf serum and antibiotics was added. The inoculated cells were cultured for 3-4 days at 37° C. and observed daily for the development of cytopathic effect (CPE). CPE was compared to the positive control (WNV inoculated cells) and negative controls (mock-inoculated cells or cells incubated with and irrelevant IgG1 only). The complete absence of CPE in an individual cell culture was defined as protection (=100% titer reduction).) The serum dilution giving protection in 66% percent of wells (i.e. two out of three wells) was defined as the 66% neutralizing antibody titer. The murine neutralising antibody 7H2 (Biorelience) was used as a positive control in the assay. A 66% neutralization concentration of ≦125 μg/ml was regarded as specific evidence of neutralizing activity of the IgG against WNV. The human anti-WNV antibodies were tested in duplicate in the virus neutralisation assay. CR4354 was an antibody showing WNV neutralizing activity. This antibody showed neutralisation in the 66% neutralizing antibody titer assay at a concentration of 0.48 μg/ml.

### Example 7

### Selection of Optimized Variants of Neutralising Monoclonal Anti-WNV Antibody CR4354

The potency and affinity of the anti-WNV monoclonal antibody called CR4354 was improved based on the following hypothesis. The specificity of CR4354 (as determined by the CDR3 region on the heavy chain variable chain) is one that targets a potent neutralising epitope of WNV, but the light chain that is randomly paired with the heavy chain (through the phage-display process) does not optimally recreate the original antigen binding site. Pairing with a more optimally mutated light chain might improve the ‘fit’ of the antibody-binding pocket for the cognate antigen. Thus, replacement of the light chain might be a way of improving the potency and affinity of the antibody.

Analysis of the heavy and light chain of antibody CR4354 showed that they belong to the VH1 1-46 (DP-7) and Vlambda1 (1c-V1-16) gene family, respectively. Analysis of the complete list of scFv selected from the WNV immune library described in Example 1 revealed 5 scFvs, i.e. SC04-261, SC04-267, SC04-328, SC04-335 and SC04-383, that had light chains having the same gene family as the light chain of CR4354. None of the scFvs or their respective IgGs showed WNV neutralizing activity. Each of these light chains contained mutations in the CDR and framework regions away from the germline indicating that they had been modified as part of the natural affinity maturation process.

In short, the construction of the antibodies went as follows. Heavy chain variable regions of the scFvs called SC04-261, SC04-267, SC04-328, SC04-335 and SC04-354 were PCR-amplified using oligonucleotides to append restriction sites and/or sequences for expression in the IgG expression vector pSyn-C18-HCγ1 and cloned into this vector. Amplification was done using the following oligonucleotide sets: SC04-261, 5H-A (SEQ ID NO:17) and sy3H-A (SEQ ID NO:18); SC04-267, 5H-A (SEQ ID NO:17) and sy3H-C (SEQ ID NO:19); SC04-328, 5H-A (SEQ ID NO:17) and sy3H-A (SEQ ID NO:18); SC04-335, 5H-C (SEQ ID NO:20) and sy3H-A (SEQ ID NO:18); and SC04-354, 5H-A (SEQ ID NO:17) and sy3H-A (SEQ ID NO:18).

The heavy chain variable region of the scFv called SC04-383 was cloned by restriction digest using the enzymes SfiI and XhoI in the IgG expression vector pIg-C911-HCgamma1.

The light chain variable region of the scFv called SC04-267 and sc04-354 was first amplified using the oligonucleotides sc04-267, 5L-C (SEQ ID NO:21) and sy3L-Amod (SEQ ID NO:22) and SC04-354, 5L-C (SEQ ID NO:21) and sy3L-C (SEQ ID NO:23) and the PCR product cloned into vector pSyn-C04-Clambda.

Light chain variable regions of the scFvs called SC04-261, SC04-328, SC04-335, and SC04-383 were cloned directly by restriction digest using the enzymes SalI and NotI for expression in the IgG expression vector pIg-C910-Clambda.

Nucleotide sequences for all constructs were verified according to standard techniques known to the skilled artisan.

The resulting expression constructs pgG104-261C18, pgG104-267C18, pgG104-328C18, pgG104-335C18, pgG104-354C18 and pgG104-383C911 encoding the anti-WNV human IgG1 heavy chains and pgG104-261C910, pgG104-267C04, pgG104-328C910, pgG104-335C910, pgG104-354C04 and pgG104-383C910 encoding the anti-WNV human IgG1 light chains were transiently expressed in combination in 293T cells and supernatants containing human IgG1 antibodies were obtained.

The nucleotide sequences of the heavy chains of the antibodies called CR4261, CR4267, CR4328, CR4335, CR4354 and CR4383 are shown in SEQ ID NOs:24, 26, 28, 30, 32 and 34, respectively (the variable regions are from nucleotides 1-348; 1-381; 1-348; 1-351; 1-363; and 1-372, respectively). The amino acid sequences of the heavy chains of the antibodies called CR4261, CR4267, CR4328, CR4335, CR4354 and CR4383 are shown in SEQ ID Nos:25, 27, 29, 31, 33, and 35, respectively (the variable regions are from amino acids 1-116; 1-127; 1-116; 1-117; 1-121; and 1-124, respectively). The nucleotide sequences of the light chain of antibodies CR4261, CR4267, CR4328, CR4335, CR4354 and CR438 are shown in SEQ ID NOs:36, 38, 40, 42, 44, and 46, respectively (the variable regions are from nucleotides 1-342; 1-330; 1-339; 1-339; 1-330; and 1-339, respectively). The amino acid sequences of the light chain of antibodies CR4261, CR4267, CR4328, CR4335, CR4354 and CR4383 are shown in SEQ ID NOs:37, 39, 41, 43, 45, and 47, respectively (the variable regions are from amino acids 1-114; 1-110; 1-113; 1-113; 1-110; and 1-113, respectively).

The expression construct encoding the heavy chain of CR4354 was combined with the constructs expressing the light chains of the respective antibodies for transfection of HEK293T cells essentially as described in Example 5. The obtained antibodies were designated CR4354L4261, CR4354L4267, CR4354L4328, CR4354L4335 and CR4354L4383. Supernatants were tested for binding by ELISA staining as described in Example 5 and for potency in the in vitro neutralization assay as described in Example 6.

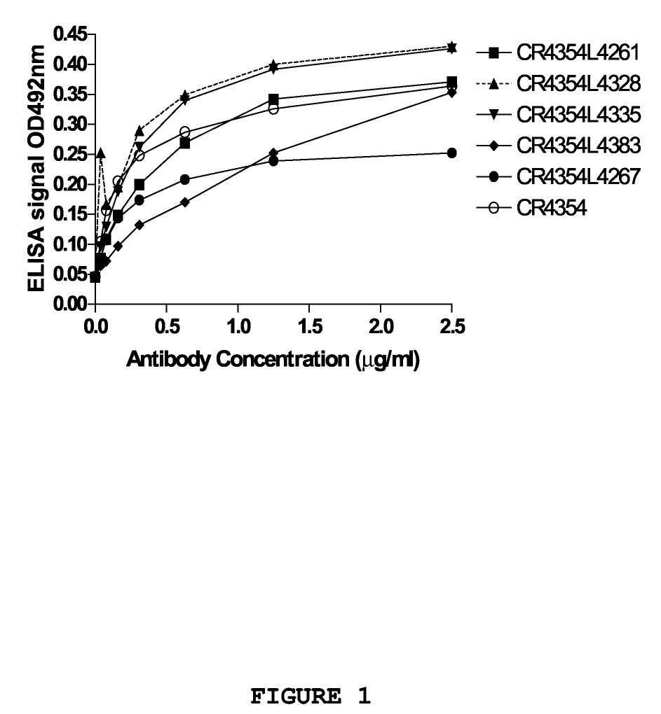

The binding data showed that all shuffled variants have specificity for the pre-selected antigen (see FIG. 1).

In terms of functional activity, it was concluded that two chain shuffled variants CR4354L4328 and CR4354L4335 had a higher affinity for WNV compared to CR4354. CR4354L4261 bound the virus with a similar affinity compared to CR4354, while both CR4354L4383 and CR4354L4267 bound with a lower affinity to the virus compared to CR4354 (see FIG. 1).

Furthermore, the antibodies CR4354L4383 and CR4354L4267 did not show any WNV neutralizing activity which was consistent with their lower binding affinity. CR4354L4261 had a neutralization endpoint concentration similar to the original antibody CR4534, again consistent with the binding data. CR4354L4335 that bound WNV with a higher affinity compared to CR4354 had a lower neutralizing activity compared to the original antibody CR4534. In contrast, the antibody variant CR4354L4328 that had a higher affinity for WNV compared to CR4354 also had a higher neutralizing activity compared to the original antibody CR4534 (see Table 8). In 4 out of 5 cases there was a direct correlation between binding affinity and neutralization potency of the variants. It was demonstrated that substituting similar light chains can improve a functionality of interest of an antibody, e.g. affinity or neutralizing activity.

TABLE 1

Human lambda chain variable region primers
(sense).

| Primer | Primer nucleotide | |
|---|---|---|
| name | sequence | SEQ ID NO |
| HuVλ1A | 5′-CAGTCTGTGCTGACT | SEQ ID NO: 48 |
| | CAGCCACC-3′ | |
| HuVλ1B | 5′-CAGTCTGTGYTGACG | SEQ ID NO: 49 |
| | CAGCCGCC-3′ | |
| HuVλ1C | 5′-CAGTCTGTCGTGACG | SEQ ID NO: 50 |
| | CAGCCGCC-3′ | |
| HuVλ2 | 5′-CARTCTGCCCTGACT | SEQ ID NO: 51 |
| | CAGCCT-3′ | |
| HuVλ3A | 5′-TCCTATGWGCTGACT | SEQ ID NO: 52 |
| | CAGCCACC-3′ | |
| HuVλ3B | 5′-TCTTCTGAGCTGACT | SEQ ID NO: 53 |
| | CAGGACCC-3′ | |
| HuVλ4 | 5′-CACGTTATACTGACT | SEQ ID NO: 54 |
| | CAACCGCC-3′ | |
| HuVλ5 | 5′-CAGGCTGTGCTGACT | SEQ ID NO: 55 |
| | CAGCCGTC-3′ | |
| HuVλ6 | 5′-AATTTTATGCTGACT | SEQ ID NO: 56 |
| | CAGCCCCA-3′ | |
| HuVλ7/8 | 5′-CAGRCTGTGGTGACY | SEQ ID NO: 57 |
| | CAGGAGCC-3′ | |
| HuVλ9 | 5′-CWGCCTGTGCTGACT | SEQ ID NO: 58 |
| | CAGCCMCC-3′ | |

TABLE 2

Human kappa chain variable region primers
(sense).

| Primer | Primer nucleotide | |
|---|---|---|
| name | sequence | SEQ ID NO |
| HUVκ1B | 5′-GACATCCAGWTGACCC | SEQ ID NO: 59 |
| | AGTCTCC-3′ | |
| HuVκ2 | 5′-GATGTTGTGATGACT | SEQ ID NO: 60 |
| | CAGTCTCC-3′ | |
| HuVκ3 | 5′-GAAATTGTGWTGACR | SEQ ID NO: 61 |
| | CAGTCTCC-3′ | |
| HUVκ4 | 5′-GATATTGTGATGACC | SEQ ID NO: 62 |
| | CACACTCC-3′ | |
| HuVκ5 | 5′-GAAACGACACTCACG | SEQ ID NO: 63 |
| | CAGTCTCC-3′ | |
| HuVκ6 | 5′-GAAATTGTGCTGACTC | SEQ ID NO: 64 |
| | AGTCTCC-3′ | |

TABLE 3

Human kappa chain variable region primers ex-
tended with SalI restriction sites (sense),
human kappa chain J-region primers extended
with NotI restriction sites (anti-sense),
human lambda chain variable region primers ex-
tended with SalI restriction sites (sense) and
human lambda chain J-region primers extended
with NotI restriction sites (anti-sense).

| | Primer nucleotide | |
|---|---|---|
| Primer name | sequence | SEQ ID NO |
| HuVκ1B-SalI | 5′-TGAGCACACAGGTCG | SEQ ID NO: 65 |
| | ACGGACATCCAGWTGACC | |
| | CAGTCTCC-3′ | |
| HuVκ2-SalI | 5′-TGAGCACACAGGTCG | SEQ ID NO: 66 |
| | ACGGATGTTGTGATGACT | |
| | CAGTCTCC-3′ | |
| HuVκ3B-SalI | 5′-TGAGCACACAGGTCG | SEQ ID NO: 67 |
| | ACGGAAATTGTGWTGACR | |
| | CAGTCTCC-3′ | |
| HuVκ4B-SalI | 5′-TGAGCACACAGGTCG | SEQ ID NO: 68 |
| | ACGGATATTGTGATGACC | |
| | CACACTCC-3′ | |
| HuVκ5-SalI | 5′-TGAGCACACAGGTCGACG | SEQ ID NO: 69 |
| | GAAACGACACTCACGCAGTCT | |
| | CC-3′ | |
| HuVκ6-SalI | 5′-TGAGCACACAGGTCG | SEQ ID NO: 70 |
| | ACGGAAATTGTGCTGACT | |
| | CAGTCTCC-3′ | |
| HUJκ1-NotI | 5′-GAGTCATTCTCGACTTGC | SEQ ID NO: 71 |
| | GGCCGCACGTTTGATTTCCAC | |
| | CTTGGTCCC-3′ | |
| HUJκ2-NotI | 5′-GAGTCATTCTCGACT | SEQ ID NO: 72 |
| | TGCGGCCGCACGTTTGAT | |
| | CTCCAGCTTGGTCCC-3′ | |
| HuJκ3-NotI | 5′-GAGTCATTCTCGACTTGC | SEQ ID NO: 73 |
| | GGCCGCACGTTTGATATCCAC | |
| | TTTGGTCCC-3′ | |
| HuJκ4-NotI | 5′-GAGTCATTCTCGACT | SEQ ID NO: 74 |
| | TGCGGCCGCACGTTTGAT | |
| | CTCCACCTTGGTCCC-3′ | |
| HuJκ5-NotI | 5′-GAGTCATTCTCGACTTGC | SEQ ID NO: 75 |
| | GGCCGCACGTTTAATCTCCAG | |
| | TCGTGTCCC-3′ | |
| HuVλ1A-SalI | 5′-TGAGCACACAGGTCGACG | SEQ ID NO: 76 |
| | CAGTCTGTGCTGACTCAGCCA | |
| | CC-3′ | |
| HuVλ1B-SalI | 5′-TGAGCACACAGGTCGACG | SEQ ID NO: 77 |
| | CAGTCTGTGYTGACGCAGCCG | |
| | CC-3′ | |
| HuVλ1C-SalI | 5′-TGAGCACACAGGTCGACG | SEQ ID NO: 78 |
| | CAGTCTGTCGTGACGCAGCCG | |
| | CC-3′ | |
| HuVλ2-SalI | 5′-TGAGCACACAGGTCGACG | SEQ ID NO: 79 |
| | CARTCTGCCCTGACTCAGCCT- | |
| | 3′ | |
| HuVλ3A-SalI | 5′-TGAGCACACAGGTCGACG | SEQ ID NO: 80 |
| | TCCTATGWGCTGACTCAGCCA | |
| | CC-3′ | |
| HuVλ3B-SalI | 5′-TGAGCACACAGGTCGACG | SEQ ID NO: 81 |
| | TCTTCTGAGCTGACTCAGGAC | |
| | CC-3′ | |
| HuVλ4-SalI | 5′-TGAGCACACAGGTCGACG | SEQ ID NO: 82 |
| | CACGTTATACTGACTCAACCG | |
| | CC-3′ | |
| HuVλ5-SalI | 5′-TGAGCACACAGGTCGACG | SEQ ID NO: 83 |
| | CAGGCTGTGCTGACTCAGCCG | |
| | TC-3′ | |
| HuVλ6-SalI | 5′-TGAGCACACAGGTCGACG | SEQ ID NO: 84 |
| | AATTTTATGCTGACTCAGCCC | |
| | CA-3′ | |
| HuVλ7/8-SalI | 5′-TGAGCACACAGGTCGACG | SEQ ID NO: 85 |
| | CAGRCTGTGGTGACYCAGGAG | |
| | CC-3′ | |
| HuVλ9-SalI | 5′-TGAGCACACAGGTCGACG | SEQ ID NO: 86 |
| | CWGCCTGTGCTGACTCAGCCM | |
| | CC-3′ | |
| HuJλ1-NotI | 5′-GAGTCATTCTCGACTTGC | SEQ ID NO: 87 |
| | GGCCGCACCTAGGACGGTGAC | |
| | CTTGGTCCC-3′ | |
| HuJλ2/3-NotI | 5′-GAGTCATTCTCGACTTGC | SEQ ID NO: 88 |
| | GGCCGCACCTAGGACGGTCAG | |
| | CTTGGTCCC-3′ | |
| HuJλ4/5-NotI | 5′-GAGTCATTCTCGACTTGC | SEQ ID NO: 89 |
| | GGCCGCACYTAAAACGGTGAG | |
| | CTGGGTCCC-3′ | |

TABLE 4
Distribution of the different light chain
products over the 10 fractions.

| Light chain | Number of | Fraction | |
|---|---|---|---|
| products | alleles | number | alleles/fraction |
| Vk1B/Jk1-5 | 19 | 1 and 2 | 9.5 |
| Vk2/Jk1-5 | 9 | 3 | 9 |
| Vk3B/Jk1-5 | 7 | 4 | 7 |
| Vk4B/Jk1-5 | 1 | 5 | 5 |
| Vk5/Jk1-5 | 1 | | |
| Vk6/Jk1-5 | 3 | | |
| Vλ1A/Jl1-3 | 5 | 6 | 5 |
| Vλ1B/Jl1-3 | | | |
| Vλ1C/Jl1-3 | | | |
| Vλ2/Jl1-3 | 5 | 7 | 5 |
| Vλ3A/Jl1-3 | 9 | 8 | 9 |
| Vλ3B/Jl1-3 | | | |
| Vλ4/Jl1-3 | 3 | 9 | 5 |
| Vλ5/Jl1-3 | 1 | | |
| Vλ6/Jl1-3 | 1 | | |
| Vλ7/8/Jl1-3 | 3 | 10 | 6 |
| Vλ9/Jl1-3 | 3 | | |

TABLE 5

Human IgG heavy chain variable region primers
(sense).

| Primer | Primer nucleotide | |
|---|---|---|
| name | sequence | SEQ ID NO |
| HuVH1B/7A | 5′-CAGRTGCAGCTGGTG | SEQ ID NO: 90 |
| | CARTCTGG-3′ | |
| HuVH1C | 5′-SAGGTCCAGCTGGTR | SEQ ID NO: 91 |
| | CAGTCTGG-3′ | |
| HuVH2B | 5′-SAGGTGCAGCTGGTG | SEQ ID NO: 92 |
| | GAGTCTGG-3′ | |
| HuVH3B | 5′-SAGGTGCAGCTGGTG | SEQ ID NO: 93 |
| | GAGTCTGG-3′ | |
| HuVH3C | 5′-GAGGTGCAGCTGGTG | SEQ ID NO: 94 |
| | GAGWCYGG-3′ | |
| HuVH4B | 5′-CAGGTGCAGCTACAG | SEQ ID NO: 95 |
| | CAGTGGGG-3′ | |
| HuVH4C | 5′-CAGSTGCAGCTGCAG | SEQ ID NO: 96 |
| | GAGTCSGG-3′ | |
| HuVH5B | 5′-GARGTGCAGCTGGTG | SEQ ID NO: 97 |
| | CAGTCTGG-3′ | |
| HuVH6A | 5′-CAGGTACAGCTGCAG | SEQ ID NO: 98 |
| | CAGTCAGG-3′ | |

TABLE 6

Human IgG heavy chain variable region primers
extended with SfiI/NcoI restriction sites
(sense) and human IgG heavy chain J-region
primers extended with XhoI/BstEII restriction
sites (anti-sense).

| | Primer nucleotide | |
|---|---|---|
| Primer name | sequence | SEQ ID NO |
| HUVH1B/7A-SfiI | 5′-GTCCTCGCAACTGCG | SEQ ID NO: 99 |
| | GCCCAGCCGGCCATGGCC | |
| | CAGRTGCAGCTGGTGCAR | |
| | TCTGG-3′ | |
| HuVH1C-SfiI | 5′-GTCCTCGCAACTGCG | SEQ ID NO: 100 |
| | GCCCAGCCGGCCATGGCC | |
| | SAGGTCCAGCTGGTRCAG | |
| | TCTGG-3′ | |
| HuVH2B-SfiI | 5′-GTCCTCGCAACTGCG | SEQ ID NO: 101 |
| | GCCCAGCCGGCCATGGCC | |
| | CAGRTCACCTTGAAGGAG | |
| | TCTGG-3′ | |
| HuVH3B-SfiI | 5′GTCCTCGCAACTGCGGCC | SEQ ID NO: 102 |
| | CAGCCGGCCATGGCCSAGGTG | |
| | CAGCTGGTGGAGTCTGG-3′ | |
| HuVH3C-SfiI | 5′-GTCCTCGCAACTGCG | SEQ ID NO: 103 |
| | GCCCAGCCGGCCATGGCC | |
| | GAGGTGCAGCTGGTGGAG | |
| | WCYGG-3′ | |
| HuVH4B-SfiI | 5′-GTCCTCGCAACTGCG | SEQ ID NO: 104 |
| | GCCCAGCCGGCCATGGCC | |
| | CAGGTGCAGCTACAGCAG | |
| | TGGGG-3′ | |
| HuVH4C-SfiI | 5′-GTCCTCGCAACTGCGGCC | SEQ ID NO: 105 |
| | CAGCCGGCCATGGCCCAGSTG | |
| | CAGCTGCAGGAGTCSGG-3′ | |
| HuVH5B-SfiI | 5′-GTCCTCGCAACTGCG | SEQ ID NO: 106 |
| | GCCCAGCCGGCCATGGCC | |
| | GARGTGCAGCTGGTGCAG | |
| | TCTGG-3′ | |
| HuVH6A-SfiI | 5′-GTCCTCGCAACTGCG | SEQ ID NO: 107 |
| | GCCCAGCCGGCCATGGCC | |
| | CAGGTACAGCTGCAGCAG | |
| | TCAGG-3′ | |
| HuJH1/2-XhoI | 5′-GAGTCATTCTCGACTCGA | SEQ ID NO: 108 |
| | GACGGTGACCAGGGTGCC-3′ | |
| HuJH3-XhoI | 5′-GAGTCATTCTCGACT | SEQ ID NO: 109 |
| | CGAGACGGTGACCATTGT | |
| | CCC-3′ | |
| HuJH4/5-XhoI | 5′-GAGTCATTCTCGACT | SEQ ID NO: 110 |
| | CGAGACGGTGACCAGGGT | |
| | TCC-3′ | |
| HuJH6-XhoI | 5′-GAGTCATTCTCGACTCGA | SEQ ID NO: 111 |
| | GACGGTGACCGTGGTCCC-3′ | |

TABLE 7
Data of the single-chain Fvs capable
of binding WNV and/or WNV E protein.

| Name | VH-germline | VL-germline |
|---|---|---|
| Sc04-255 | 1-69 (DP-10) | Vl 2 (2a2 - V1-04) |
| Sc04-256 | 1-69 (DP-10) | Vl 2 (2c - V1-02) |
| Sc04-258 | 1-24 (DP-5) | Vl 3 (2e - V1-03) |
| Sc04-259 | 1-69 (DP-10) | Vl 2 (2c - V1-02) |
| Sc04-260 | 1-03 (DP-25) | Vl 1 - (1b -V1-19) |
| Sc04-261 | 1-03 (DP-25) | Vl 1 (1c -V1-16) |
| Sc04-262 | 1-02 (DP-75) | Vl 1 - (1b -V1-19) |
| Sc04-263 | 1-18 (DP-14) | Vl 1 - (1b -V1-19) |
| Sc04-264 | 1-02 (DP-75) | Vl 1 - (1b -V1-19) |
| Sc04-265 | 1-02 (DP-75) | Vl 1 (1g - V1-17) |
| Sc04-266 | 1-02 (DP-75) | Vl 1 (1g - V1-17) |
| Sc04-267 | 1-69 (DP-10) | Vl 1 (1c -V1-16) |
| Sc04-268 | 4-04 | Vl 3 (3h - V2-14) |
| Sc04-269 | 4-39 (DP-79) | Vl 3 (2e - V1-03) |
| Sc04-270 | 4-39 (DP-79) | Vl 1 - (1b -V1-19) |
| Sc04-271 | 5-51 (DP-73) | Vl 2 (2a2 - V1-04) |
| Sc04-272 | 5-51 (DP-73) | Vl 2 (2a2 - V1-04) |
| Sc04-273 | 5-51 (DP-73) | Vl 3 (3r - V2-01) |
| Sc04-274 | 5-51 (DP-73) | Vk IV (B3 - DPK24) |
| Sc04-277 | 1-46 (DP-7) | Vl 3 (3h - V2-14) |
| Sc04-278 | 4-59 (DP-71) | Vk III (A27 - DPK22) |
| Sc04-279 | 4-39 (DP-79) | Vk IV (B3 - DPK24) |
| Sc04-281 | 3-30 (DP-49) | Vk III (A27 - DPK22) |
| Sc04-282 | 5-51 (DP-73) | Vk I (L12) |
| Sc04-283 | 5-51 (DP-73) | Vk I (L12) |
| Sc04-284 | 5-51 (DP-73) | Vk III (A27 - DPK22) |
| Sc04-285 | 5-51 (DP-73) | Vk I (O12/O2 - DPK9) |
| Sc04-286 | 5-51 (DP-73) | Vk I (L12) |
| Sc04-287 | 5-51 (DP-73) | Vk IV (B3 - DPK24) |
| Sc04-288 | 5-51 (DP-73) | Vk IV (B3 - DPK24) |
| Sc04-289 | 5-51 (DP-73) | Vk III (L2 - DPK21) |
| Sc04-290 | 1-46 (DP-7) | Vk III (A27 - DPK22) |
| Sc04-292 | 1-02 (DP-75) | Vk I (O12/O2 - DPK9) |
| Sc04-293 | 1-46 (DP-7) | Vk I (O12/O2 - DPK9) |
| Sc04-294 | 1-46 (DP-7) | Vk I (O12/O2 - DPK9) |
| Sc04-295 | 1-03 (DP-25) | Vk I (O12/O2 - DPK9) |
| Sc04-296 | 1-08 (DP-15) | Vk I (O18/O8 - DPK1) |
| Sc04-297 | 1-18 (DP-14) | Vl 3 (3l - V2-13) |
| Sc04-298 | 1-18 (DP-14) | Vk I (O12/O2 - DPK9) |
| Sc04-299 | 3-30 (DP-49) | Vl 1 (1a - V1-11) |
| Sc04-300 | 3-30 (DP-49) | Vl 3 (3r - V2-01) |
| Sc04-301 | 3-30 (DP-49) | Vl 3 (2e - V1-03) |
| Sc04-302 | 3-30 (DP-49) | Vl 6 (6a - V1-22) |
| Sc04-303 | 3-30 (DP-49) | Vl 3 (3h - V2-14) |
| Sc04-304 | 3-30 (DP-49) | Vl 1 - (1b -V1-19) |
| Sc04-305 | 3-30 (DP-49) | Vl 3 (3h - V2-14) |
| Sc04-306 | 3-30 (DP-49) | Vl 3 (3h - V2-14) |
| Sc04-307 | 3-23 (DP-47) | Vl 3 (3h - V2-14) |
| Sc04-308 | 3-53 (DP-42) | Vl 3 (3r - V2-01) |
| Sc04-310 | 3-30 (DP-49) | Vl 3 (2e - V1-3) |
| Sc04-311 | 3-30 (DP-49) | Vl 1 - (1b -V1-19) |
| Sc04-312 | 3-64 | Vl 2 (2c - V1-02) |
| Sc04-313 | 3-64 | Vl 3 (2e - V1-03) |
| Sc04-315 | 3-09 (DP-31) | Vl 2 (2c - V1-02) |
| Sc04-316 | 3-09 (DP-31) | Vl 2 (2c - V1-02) |
| Sc04-317 | 3-30 (DP-49) | Vl 3 (3l - V2-13) |
| Sc04-318 | 3-23 (DP-47) | Vl 3 (3l - V2-13) |
| Sc04-319 | 3-23 (DP-47) | Vl 3 (3l - V2-13) |
| Sc04-320 | 3-11 (DP-35) | Vl 3 (3l - V2-13) |
| Sc04-321 | 5-51 (DP-73) | Vl 3 (3l - V2-13) |
| Sc04-322 | 5-51 (DP-73) | Vl 3 (3l - V2-13) |
| Sc04-323 | 3-30 (DP-49) | Vl 3 (2e - V1-3) |
| Sc04-324 | 4-31 (DP-65) | Vk I (O12/O2 - DPK9) |
| Sc04-325 | 1-69 (DP-10) | Vk IV (B3 - DPK24) |
| Sc04-326 | 1-02 (DP-75) | Vl 1 (1g - V1-17) |
| Sc04-327 | 1-03 (DP-25) | Vl 2 (2c - V1-02) |
| Sc04-328 | 1-03 (DP-25) | Vl 1 (1c -V1-16) |
| Sc04-329 | 1-02 (DP-75) | Vl 1 - (1b -V1-19) |
| Sc04-330 | 1-69 (DP-10) | Vl 2 (2b2 - V1-7) |
| Sc04-331 | 3-23 (DP-47) | Vl 3 (2e - V1-3) |
| Sc04-332 | 3-15 (DP-38) | Vl 3 (2e - V1-3) |
| Sc04-333 | 3-07 (DP-54) | Vl 2 (2c - V1-02) |
| Sc04-334 | 3-07 (DP-54) | Vl 2 (2a2 - V1-04) |
| Sc04-335 | 3-30 (DP-49) | Vl 1 (1c -V1-16) |
| Sc04-336 | 3-30 (DP-49) | Vl 3 (3l - V2-13) |
| Sc04-337 | 3-30 (DP-49) | Vl 1 (1a - V1-11) |
| Sc04-338 | 3-30 (DP-49) | Vl 1 - (1b -V1-19) |
| Sc04-339 | 3-07 (DP-54) | Vl 1 (1a - V1-11) |
| Sc04-340 | 3-23 (DP-47) | Vl 1 (1e - V1-13) |
| Sc04-341 | 4-31 (DP-65) | Vl 1 - (1b -V1-19) |
| Sc04-342 | 4-04 | Vl 1 (1e - V1-13) |
| Sc04-343 | 3-23 (DP-47) | Vl 3 (3h - V2-14) |
| Sc04-344 | 1-18 (DP-14) | Vl 3 (3l - V2-13) |
| Sc04-345 | 1-18 (DP-14) | Vl 3 (3l - V2-13) |
| Sc04-346 | 5-51 (DP-73) | Vk III (A27 - DPK22) |
| Sc04-347 | 1-02 (DP-75) | Vl 3 (3l - V2-13) |
| Sc04-348 | 3-09 (DP-31) | Vk I (O12/O2 - DPK9) |
| Sc04-351 | 1-46 (DP-7) | Vl 3 (3r - V2-01) |
| Sc04-352 | 5-51 (DP-73) | Vk I (L8 - DPK8) |
| Sc04-353 | 3-30 (DP-49) | Vk III (A27 - DPK22) |
| Sc04-354 | 1-46 (DP-7) | Vl 1 (1c -V1-16) |
| Sc04-355 | 3-30 (DP-49) | Vk III (L2 - DPK21) |
| Sc04-356 | 3-53 (DP-42) | Vl 3 (3r - V2-01) |
| Sc04-357 | 3-23 (DP-47) | Vl 2 (2c - V1-02) |
| Sc04-358 | 1-46 (DP-7) | Vk III (A27 - DPK22) |
| Sc04-359 | 3-15 (DP-38) | Vl 7 (7b - V3-03) |
| Sc04-360 | 3-11 (DP-35) | Vl 3 (3r - V2-01) |
| Sc04-361 | 5-51 (DP-73) | Vk IV (B3 - DPK24) |
| Sc04-363 | 3-11 (DP-35) | Vl 1 (1e - V1-13) |
| Sc04-364 | 4-39 (DP-79) | Vl 1 - (1b -V1-19) |
| Sc04-365 | 6-01 (DP-74) | Vl 7 (7a - V3-02) |
| Sc04-368 | 2-05 | Vl 1 (1e - V1-13) |
| Sc04-370 | 5-51 (DP-73) | Vl 2 (2a2 - V1-04) |
| Sc04-371 | 3-66 | Vl 2 (2a2 - V1-04) |
| Sc04-372 | 1-03 (DP-25) | Vl10 (10a - V1-20) |
| Sc04-373 | 3-30 (DP-49) | Vk III (A27 - DPK22) |
| Sc04-374 | 2-05 | Vl 1 (1e - V1-13) |
| Sc04-375 | 2-05 | Vl 1 (1e - V1-13) |
| Sc04-376 | 1-02 (DP-75) | Vk I (O12/O2 - DPK9) |
| Sc04-377 | 5-51 (DP-73) | Vl 3 (3h - V2-14) |
| Sc04-378 | 3-09 (DP-31) | Vk III (L25 - DPK23) |
| Sc04-379 | 3-15 (DP-38) | Vl 7 (7b - V3-03) |
| Sc04-380 | 1-69 (DP-10) | Vk IV (B3 - DPK24) |
| Sc04-381 | 4-04 | Vl 3 (3h - V2-14) |
| Sc04-382 | 5-51 (DP-73) | Vk IV (B3 - DPK24) |
| Sc04-383 | 1-02 (DP-75) | Vl 1 (1c -V1-16) |
| Sc05-001 | 1-02 (DP-1) | Vl 1 (1a - V1-11) |
| Sc05-002 | 1-02 (DP-1) | Vk III (A11 - DPK20) |
| Sc05-003 | 4-0rC15 (DP-69) | Vk III (A27 - DPK22) |
| Sc05-004 | 4-0rC15 (DP-69) | Vk III (L2 - DPK21) |
| Sc05-005 | 4-0rC15 (DP-69) | Vk III (A27 - DPK22) |
| Sc05-006 | 4-0rC15 (DP-69) | Vk III (A27 - DPK22) |
| Sc05-007 | 4-0rC15 (DP-69) | Vk III (A27 - DPK22) |
| Sc05-008 | 4-0rC15 (DP-69) | Vk III (A27 - DPK22) |
| Sc05-009 | 4-0rC15 (DP-69) | Vk III (A27 - DPK22) |
| Sc05-010 | 4-0rC15 (DP-69) | Vk I (L12) |
| Sc05-011 | 3-64 | Vl 3 (2e - V1-03) |
| Sc05-012 | 3-64 | Vl 1 (1e - V1-13) |
| Sc05-013 | 3-09 (DP-31) | Vl 2 (2c - V1-02) |
| Sc05-014 | 4-0rC15 (DP-69) | Vl 1 (1e - V1-13) |
| Sc05-015 | 3-33 (DP-50) | Vl 1 (1a - V1-11) |
| Sc05-016 | 3-33 (DP-50) | Vk I (O12/O2 - DPK9) |
| Sc05-017 | 4-04 | Vl 3 (3j - V2-06) |
| Sc05-018 | 1-02 (DP-1) | Vl 1 (1e - V1-13) |
| Sc05-019 | 1-46 (DP-7) | Vl 3 (2e - V1-03) |
| Sc05-020 | 1-69 (DP-10) | Vk I (O12/O2 - DPK9) |
| Sc05-021 | 1-02 (DP-8) | Vl 1 (1a - V1-11) |

TABLE 8
Percentage difference in 66% neutralization concentration
of IgG1 variants of CR4354 against WNV as measured by VNA.

| Antibody | Potency (%)* |
|---|---|
| CR4354 | 100 |
| CR4354L4261 | 106 |
| CR4354L4267 | Below detection |
| CR4354L4328 | 286 |
| CR4354L4335 | 60 |
| CR4354L4383 | Below detection |

*Potency is represented in comparison to original antibody CR4354 (the 66% neutralising concentration of which was set at 100%) and was calculated by dividing the 66% neutralising concentration (in μg/ml) of CR4354 by the 66% neutralising concentration (in μg/ml) of the chain shuffled variants and multiplying the resulting number by 100%.

### REFERENCES

Boel E, Verlaan S, Poppelier M J, Westerdaal N A, Van Strijp J A and Logtenberg T (2000), Functional human monoclonal antibodies of all isotypes constructed from phage display library-derived single-chain Fv antibody fragments. J. Immunol. Methods 239:153-166.
Burton D R and Barbas C F (1994), Human antibodies from combinatorial libraries. Adv. Immunol. 57:191-280.
Clackson T, Hoogenboom H R, Griffiths A D and Winter G (1991), Making antibody fragments using phage display libraries. Nature, 352:624-628.
De Kruif J, Terstappen L, Boel E and Logtenberg T (1995a), Rapid selection of cell subpopulation-specific human monoclonal antibodies from a synthetic phage antibody library. Proc. Natl. Acad. Sci. USA 92:3938.
De Kruif J, Boel E and Logtenberg T (1995b), Selection and application of human single-chain Fv antibody fragments from a semi-synthetic phage antibody display library with designed CDR3 regions. J. Mol. Biol. 248:97-105.
Huls G, Heijnen I J, Cuomo E, van der Linden J, Boel E, van de Winkel J and Logtenberg T (1999), Antitumor immune effector mechanisms recruited by phage display-derived fully human IgG1 and IgA1 monoclonal antibodies. Cancer Res. 59: 5778-5784.
Marks J D, Griffiths A D, Malmqvist M, Clackson T, Bye J M, and Winter G (1992) Bypassing immunisation: high affinity human antibodies by chain shuffling. Bio/Technology 10:779-783.

1.-20. (canceled)

21. A method of obtaining an immunoglobulin molecule with specificity for a pre-selected antigen having a functionality of interest, wherein the functionality of interest is other than binding specificity, the method comprising the steps of:
a) isolating a first nucleic acid molecule encoding first immunoglobulin molecule's heavy chain, the first immunoglobulin molecule having specificity for the pre-selected antigen and a functionality of interest,
b) transfecting a host with the first nucleic acid molecule and a second nucleic acid molecule encoding the light chain of a second immunoglobulin molecule,
c) culturing the host under conditions conducive to the expression of a third immunoglobulin molecule, the third immunoglobulin molecule comprising the heavy chain of the first immunoglobulin molecule and the light chain of the second immunoglobulin molecule,
d) determining whether the third immunoglobulin molecule still has specificity for the pre-selected antigen,
e) determining the functionality of interest of the third immunoglobulin molecule and comparing it with the functionality of interest of the first immunoglobulin molecule, wherein steps d) and e) can be in either order or simultaneously, and
f) selecting a third immunoglobulin molecule having an improved functionality of interest and still having specificity for the pre-selected antigen
wherein the functionality of interest is selected from the group consisting of affinity for the pre-selected antigen, neutralizing activity, opsonic activity, complement fixing activity, recruitment and attachment of immune effector cells, and any combination thereof.

22. The method according to claim 21, wherein the pre-selected antigen is from an organism selected from the group consisting of a virus, a protozoa, a bacterium, a yeast, a fungus and a parasite.

23. The method according to claim 21, wherein the method further comprises the step of recovering the expressed third immunoglobulin molecule after step c.

24. The method according to claim 21, wherein the light chain of the first immunoglobulin molecule and the light chain of the second immunoglobulin molecule are members of the same gene family and/or the heavy chain of the first immunoglobulin molecule and the heavy chain of the second immunoglobulin molecule are members of the same gene family.

25. The method according to claim 24, wherein the light chain of the first immunoglobulin molecule and the light chain of the second immunoglobulin molecule are members of the same germline and/or the heavy chain of the first immunoglobulin molecule and the heavy chain of the second immunoglobulin molecule are members of the same germline.

26. The method according to claim 21, wherein the first immunoglobulin molecule is obtained from a collection of binding molecules displayed on the surface of replicable genetic display packages.

27. The method according to claim 26, wherein the replicable genetic package is selected from the group consisting of phages, bacteriophages, bacteria, yeasts, fungi, viruses, and spores of a microorganism.

28. The method according to claim 21, wherein the first immunoglobulin molecule is obtained from a collection of binding molecules displayed by means of ribosome display, mRNA display, and/or CIS display.

29. The method according to claim 21, wherein the first immunoglobulin molecule and the second immunoglobulin molecule are both from one or more pools of immunoglobulin molecules selected against the pre-selected antigen.

30. The method according to claim 27, wherein the collection of binding molecules is prepared from RNA isolated from cells obtained from a subject that has been vaccinated or exposed to an infectious agent.

31. The method according to claim 30, wherein the infectious agent is a virus, a protozoan, a bacterium, yeast, a fungus or a parasite.

32. The method according to claim 21, wherein the first immunoglobulin molecule and the second immunoglobulin molecule each have a functionality of interest.

33. The method according to claim 21, wherein the first, second, and third immunoglobulins are human.

34. The method according to claim 21, wherein the first nucleic acid molecule encoding and the second nucleic acid molecule are expressed from separate expression vectors.

35. The method according to claim 21, wherein the first nucleic acid molecule and the second nucleic acid molecule are expressed from a single expression vector.

36. The method according to claim 21, wherein the first, second and third immunoglobulin molecule are selected from the group consisting of IgA, IgD, IgE, IgG, and IgM.
